(19) United States
(12) Patent Application Publication
(10) Pub. No.: US 2024/0138713 A1
(43) Pub. Date: May 2, 2024

(54) HARMONIC INDEX ESTIMATION DEVICE, ESTIMATION SYSTEM, HARMONIC INDEX ESTIMATION METHOD, AND RECORDING MEDIUM

(71) Applicant: NEC Corporation, Tokyo (JP)

(72) Inventors: Chenhui HUANG, Tokyo (JP); Zhenwei WANG, Tokyo (JP); Fumiyuki NIHEY, Tokyo (JP)

(73) Assignee: NEC Corporation, Tokyo (JP)

(21) Appl. No.: 18/410,260

(22) Filed: Jan. 11, 2024

Related U.S. Application Data
(63) Continuation of application No. 18/203,334, filed on May 30, 2023.

(30) Foreign Application Priority Data
Jun. 8, 2022 (JP) 2022-092757

(51) Int. Cl. A61B 5/11; A61B 5/00

(52) U.S. Cl. CPC A61B 5/112; A61B 5/6807

(57) ABSTRACT

Provided is a harmonic index estimation device including a communication unit that acquires feature amount data including a feature amount used for estimation of a harmonic index related to smoothness of movement of a waist, the feature amount being extracted from a gait waveform of a spatial acceleration and a spatial angular velocity included in sensor data related to movement of a foot of a subject, a storage unit that stores an estimation model that outputs an estimation value related to the harmonic index, according to an input of the feature amount included in the feature amount data, an estimation unit that inputs the feature amount included in the feature amount data to the estimation model and estimate a harmonic index of the subject according to the estimation value output from the estimation model, and an output unit that outputs information associated to the harmonic index.

This application is a Continuation of U.S. application Ser. No. 18/203,334 filed on May 30, 2023, which is based upon and claims the benefit of priority from Japanese Patent Application No. 2022-092757, filed on Jun. 8, 2022, the disclosure of which is incorporated herein in its entirety by reference.

### TECHNICAL FIELD

The present disclosure relates to a harmonic index estimation device and the like for estimating a harmonic index related to smoothness of movement of a waist.

### BACKGROUND ART

With growing interest in healthcare, services that provide information corresponding to a gait have attracted attention. For example, a technique for analyzing a gait using sensor data measured by a sensor mounted on footwear such as shoes has been developed. Features associated with a gait event related to physical conditions appear in the time-series data of the sensor data. The physical conditions of the subject can be estimated by analyzing the gait data including the features associated with the gait event. The harmonic index (Harmonic Ratio) related to the smoothness of the movement of the waist during walking is an index indicating the shake and movement of the waist. If the harmonic index can be estimated with high accuracy by analyzing the gait data, a service according to the need for healthcare can be provided.

Patent Literature 1 (JP 2020-151470 A) discloses a walking evaluation device that evaluates walking ability of a user. The device of Patent Literature 1 calculates a plurality of gait indices related to a walking state using a plurality of pieces of gait data acquired from a subject. In the method of Patent Literature 1, a gait score of a subject is calculated using the gait data acquired by an acceleration sensor attached to the waist of the subject. In the method of Patent Literature 1, a harmonic index (Harmonic Ratio), which is one of gait indices, is calculated from acceleration waveforms in a vertical direction, a lateral direction, and a front-back direction measured by an acceleration sensor attached to a waist of a subject.

Patent Literature 2 (WO 2022/038664 A1) discloses a calculation device that calculates step lengths of both right and left feet using sensor data based on movement of a foot measured by a sensor installed on a foot portion of a pedestrian. The device of Patent Literature 2 calculates the step lengths of the left and right feet according to the gait event timing appearing in the gait waveform of the traveling-direction acceleration and the traveling direction trajectory.

Patent Literature 3 (JP 2020-144115 A) discloses a system that monitors a rhythmic movement of a person based on a signal transmitted and received via a wireless multipath channel. Patent Literature 3 discloses that a gait of a person is recognized as a rhythmic movement.

In the method of Patent Literature 1, one of indices related to smoothness of movement of the waist is measured using acceleration measured by an acceleration sensor attached to the waist. In daily life, sensors worn on the waist can limit free actions. If the position of the attached sensor deviates, the measurement accuracy decreases. Therefore, in the method of Patent Literature 1, it is not possible to easily measure the index related to the smoothness of the movement of the waist with high accuracy in daily life.

In the method of Patent Literature 2, the step lengths of both left and right feet are calculated using sensor data based on the movement of the foot. Patent Literature 2 does not disclose estimating a harmonic index using sensor data based on movement of a foot.

In the method of Patent Literature 3, a rhythmic movement such as a gait of a person is recognized based on a signal transmitted and received via a wireless multipath channel. Therefore, in the method of Patent Literature 3, the gait cannot be recognized unless the wireless multipath channel can be used.

An object of the present disclosure is to provide a harmonic index estimation device and the like that can easily estimate a harmonic index related to smoothness of movement of a waist with high accuracy in daily life.

### SUMMARY

A harmonic index estimation device according to an aspect of the present disclosure includes a communication unit that acquires feature amount data including a feature amount to be used for estimation of a harmonic index related to smoothness of movement of a waist, the feature amount being extracted from a gait waveform of a spatial acceleration and a spatial angular velocity included in sensor data related to movement of a foot of a subject, a storage unit that stores an estimation model that outputs an estimation value related to the harmonic index according to an input of the feature amount included in the feature amount data, an estimation unit that inputs the feature amount included in the acquired feature amount data to the estimation model and estimate a harmonic index of the subject according to the estimation value related to the harmonic index output from the estimation model, and an output unit that outputs information associated to the harmonic index of the subject.

A harmonic index estimation method according to one aspect of the present disclosure includes acquiring feature amount data including a feature amount to be used for estimation of a harmonic index related to smoothness of movement of a waist, the feature amount being extracted from a gait waveform of a spatial acceleration and a spatial angular velocity included in sensor data related to movement of a foot of a subject, storing an estimation model that outputs an estimation value related to the harmonic index according to an input of the feature amount included in the feature amount data, inputting the feature amount included in the acquired feature amount data to the estimation model and estimating a harmonic index of the subject according to the estimation value related to the harmonic index output from the estimation model, and outputting information associated to the harmonic index of the subject.

A program according to one aspect of the present disclosure causes a computer to execute processing of acquiring feature amount data including a feature amount to be used for estimation of a harmonic index related to smoothness of movement of a waist, the feature amount being extracted from a gait waveform of a spatial acceleration and a spatial angular velocity included in sensor data related to movement of a foot of a subject, processing of storing an estimation model that outputs an estimation value related to the harmonic index according to an input of the feature amount included in the feature amount data, processing of inputting the feature amount included in the acquired feature amount data to the estimation model and estimating a harmonic index of the subject according to the estimation value related to the harmonic index output from the estimation model, and processing of outputting information associated to the harmonic index of the subject.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of an estimation system according to a first example embodiment;

FIG. 2 is a block diagram illustrating an example of a configuration of a measurement device included in the estimation system according to the first example embodiment;

FIG. 3 is a conceptual diagram illustrating an arrangement example of the measurement device according to the first example embodiment;

FIG. 4 is a conceptual diagram for explaining an example of a relationship between a local coordinate system and a global coordinate system set in the measurement device according to the first example embodiment;

FIG. 5 is a conceptual diagram for explaining a human body surface used in the description of the measurement device according to the first example embodiment;

FIG. 6 is a conceptual diagram for explaining a gait cycle used in the description of a gait measurement device according to the first example embodiment;

FIG. 7 is a graph for explaining an example of time-series data of sensor data measured by the measurement device according to the first example embodiment;

FIG. 8 is a diagram for explaining an example of normalization of gait waveform data extracted from time-series data of sensor data measured by the measurement device according to the first example embodiment;

FIG. 9 is a conceptual diagram for explaining a feature amount cluster from which a feature amount data generation unit of the measurement device according to the first example embodiment extracts a feature amount;

FIG. 10 is a block diagram illustrating an example of a configuration of a harmonic index estimation device included in the estimation system according to the first example embodiment;

FIG. 11 is a conceptual diagram for explaining learning of the estimation model used by the harmonic index estimation device included in the estimation system according to the first example embodiment;

FIG. 12 is a conceptual diagram for explaining estimation using an estimation model by the harmonic index estimation device included in the estimation system according to the first example embodiment;

FIG. 13 is a flowchart for explaining an example of the operation of the measurement device included in the estimation system according to the first example embodiment;

FIG. 14 is a flowchart for explaining an example of the operation of the harmonic index estimation device included in the estimation system according to the first example embodiment;

FIG. 15 is a conceptual diagram for explaining an application example of the estimation system according to the first example embodiment;

FIG. 16 is a conceptual diagram for explaining an application example of the estimation system according to the first example embodiment;

FIG. 17 is a block diagram illustrating an example of a configuration of an estimation system according to a second example embodiment;

FIG. 18 is a block diagram illustrating an example of a configuration of a harmonic index estimation device included in an estimation system according to the second example embodiment;

FIG. 19 is a conceptual diagram for explaining learning of an estimation model used by the harmonic index estimation device included in the estimation system according to the second example embodiment;

FIG. 20 is a graph illustrating an example of time-series data of vertical-direction acceleration of the waist;

FIG. 21 is a graph illustrating an example of time-series data of the traveling-direction acceleration of the waist;

FIG. 22 is a graph illustrating an example of time-series data of the left-right direction acceleration of the waist;

FIG. 23 is a conceptual diagram for explaining estimation using an estimation model by the harmonic index estimation device included in the estimation system according to the second example embodiment;

FIG. 24 is a conceptual diagram for explaining calculation of a harmonic index by the harmonic index estimation device included in the estimation system according to the second example embodiment;

FIG. 25 is a table summarizing an example of input data to be used for estimation of a harmonic index in the vertical direction by the harmonic index estimation device included in the estimation system according to the second example embodiment;

FIG. 26 is a table summarizing an example of input data to be used for estimation of a harmonic index in the traveling direction by the harmonic index estimation device included in the estimation system according to the second example embodiment;

FIG. 27 is a table summarizing an example of input data to be used for estimation of the harmonic index in the left-right direction by the harmonic index estimation device included in the estimation system according to the second example embodiment;

FIG. 28 is a flowchart for explaining an example of the operation of the harmonic index estimation device included in the estimation system according to the second example embodiment;

FIG. 29 is a flowchart for explaining another example related to the operation of the harmonic index estimation device included in the estimation system according to the second example embodiment;

FIG. 30 is a block diagram illustrating an example of a configuration of a harmonic index estimation device according to a third example embodiment; and

FIG. 31 is a block diagram illustrating an example of a hardware configuration that executes processing of each example embodiment.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

First, an estimation system according to a first example embodiment will be described with reference to the drawings. The estimation system according to the present example embodiment measures sensor data related to movement of a foot according to a gait of a user using a measurement device mounted on footwear. The estimation system according to the present example embodiment estimates a harmonic index related to smoothness of movement of the waist using the measured sensor data. The harmonic index is also an index of harmonicity of walking.

In the present example embodiment, an example of estimating a harmonic ratio (hereinafter, abbreviated as HR) of the waist as the harmonic index will be described. HR is an index focusing on the fact that one gait cycle is established by acceleration changes in two cycles including one cycle of one step of the right foot and one cycle of one step of the left foot. HR is calculated using frequency components obtained by performing Fourier transform on time-series data of acceleration of the waist in one gait cycle. The frequency components include even-numbered (even Harmonics) frequency components corresponding to elements during the gait cycle and odd-numbered (odd Harmonics) frequency components deviating from the even-numbered (even Harmonics) frequency components. The even-numbered frequency components are also referred to as even components. The odd-numbered frequency components are also referred to as odd components.

The method of calculating HR varies depending on the direction during walking. HR in the vertical direction and the traveling direction is a ratio of a power sum of even components and a power sum of odd components. On the other hand, HR in the left-right direction is a ratio of the power sum of odd components and the power sum of even components since one step (two steps) of the left and right feet in one gait cycle is one cycle.

HR (VT) in the vertical direction and HR (AP) in the traveling direction are calculated using Equation 1 below.

$$HR ( VT ) , HR ( AP ) = \frac{\sum even harmonics}{\sum odd harmonics} \qquad ( 1 )$$

HR (ML) in the left-right direction is calculated using Equation 2 below.

$$HR ( ML ) = \frac{\sum odd harmonics}{\sum even harmonics} \qquad ( 2 )$$

The denominator/numerator of Equations 1 and 2 are the power sum of the even components or the odd components in one gait cycle.

The left and right feet are connected to the pelvis through the lower thigh and the thigh. Hip and knee joints are located between the left and right feet and the pelvis, but the periodicity of the pelvis and the waist during walking is similar. Therefore, there is a phase in which the movement of the left and right feet and the movement of the waist interlock with each other. In the present example embodiment, the harmonic index related to the smoothness of the movement of the waist is estimated using the sensor data related to the movement of the foot.

### Configuration

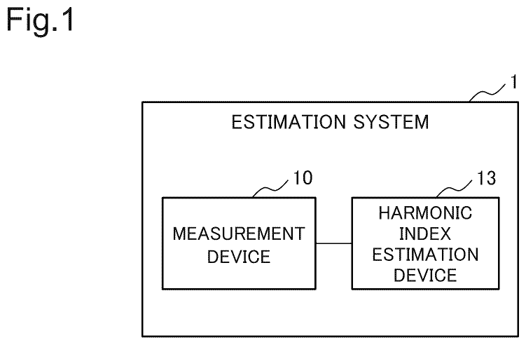

FIG. 1 is a block diagram illustrating an example of a configuration of an estimation system 1 according to the present example embodiment. The estimation system 1 includes a measurement device 10 and a harmonic index estimation device 13. In the present example embodiment, an example in which the measurement device 10 and the harmonic index estimation device 13 are configured as separate pieces of hardware will be described. For example, the measurement device 10 is installed on footwear of a subject (user) who is an estimation target of the harmonic index. For example, the function of the harmonic index estimation device 13 is installed in a mobile terminal carried by a subject (user). Hereinafter, configurations of the measurement device 10 and the harmonic index estimation device 13 will be individually described.

### Measurement Device

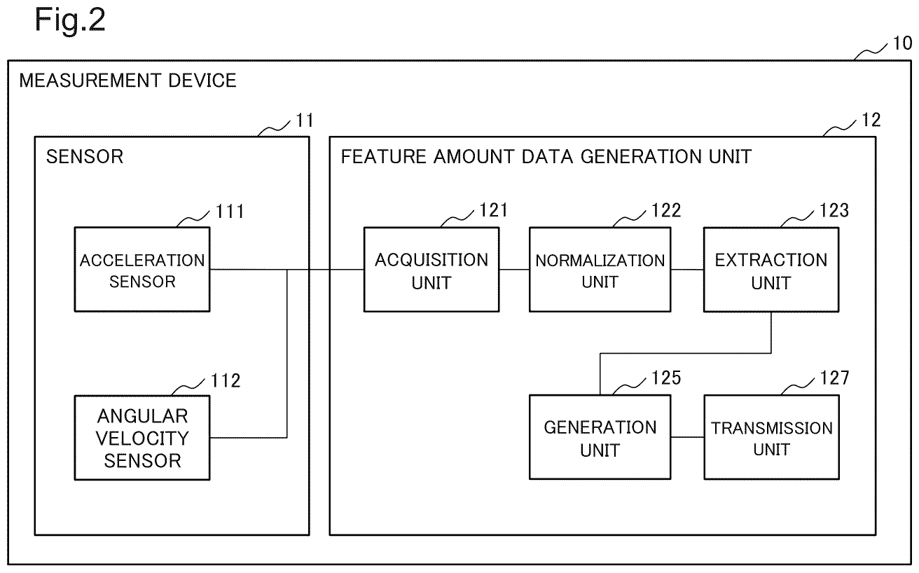

FIG. 2 is a block diagram illustrating an example of a configuration of the measurement device 10. The measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include sensors other than the acceleration sensor 111 and the angular velocity sensor 112. Sensors other than the acceleration sensor 111 and the angular velocity sensor 112 that can be included in the sensor 11 will not be described.

The acceleration sensor 111 is a sensor that measures accelerations (also referred to as spatial accelerations) in three axial directions. The acceleration sensor 111 measures acceleration (also referred to as spatial acceleration) as a physical quantity related to the movement of the foot. The acceleration sensor 111 outputs the measured acceleration to the feature amount data generation unit 12. For example, a sensor of a piezoelectric type, a piezoresistive type, a capacitance type, or the like can be used as the acceleration sensor 111. The measurement method of the sensor used as the acceleration sensor 111 is not particularly limited as long as the sensor can measure acceleration.

The angular velocity sensor 112 is a sensor that measures an angular velocity (also referred to as a spatial angular velocity) about three axes. The angular velocity sensor 112 measures an angular velocity (also referred to as a spatial angular velocity) as a physical quantity related to the movement of the foot. The angular velocity sensor 112 outputs the measured angular velocity to the feature amount data generation unit 12. For example, a sensor of a vibration type, a capacitance type, or the like can be used as the angular velocity sensor 112. The measurement method of the sensor used as the angular velocity sensor 112 is not particularly limited as long as the sensor can measure an angular velocity.

The sensor 11 is, for example, an inertial measurement device that measures acceleration and angular velocity. An example of the inertial measurement device is an inertial measurement unit (IMU). The IMU includes the acceleration sensor 111 that measures accelerations in three axial directions and the angular velocity sensor 112 that measures angular velocities about the three axes. The sensor 11 may be an inertial measurement device such as a vertical gyro (VG) or an attitude heading reference system (AHRS). The sensor 11 may be GPS/INS (Global Positioning System/Inertial Navigation System). The sensor 11 may be a device other than the inertial measurement device as long as it can measure a physical quantity related to the movement of the foot.

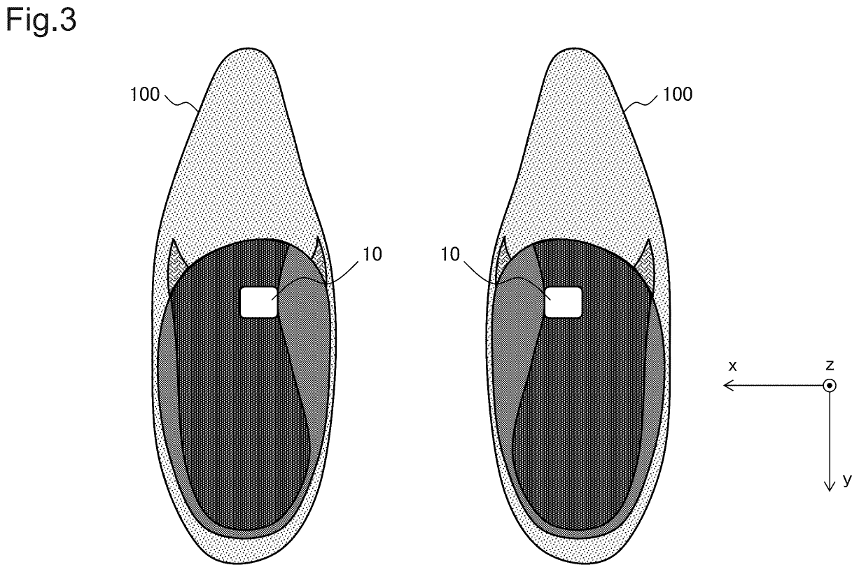

FIG. 3 is a conceptual diagram illustrating an example in which the measurement device 10 is arranged in the shoes 100 of both feet. In the example of FIG. 3, the measurement device 10 is installed at a position corresponding to the back side of the arch of foot. The measurement device 10 may be installed at a position other than the back side of the arch of the foot as long as the sensor data related to the movement of the foot can be measured. For example, the measurement device 10 is disposed in an insole inserted into the shoe 100. The measurement device 10 may be disposed on the bottom surface of the shoe 100. The measurement device 10 may be embedded in the main body of the shoe 100. The measurement device 10 may be detachable from the shoe 100 or may not be detachable from the shoe 100. The measurement device 10 may be installed on a sock worn by the user or a decorative article such as an anklet worn by the user. The measurement device 10 may be directly attached to the foot or may be embedded in the foot. FIG. 3 illustrates an example in which the measurement device 10 is installed on the shoes 100 of both feet. The measurement device 10 may be installed on the shoe 100 of one foot.

In the example of FIG. 3, a local coordinate system including the x-axis in the left-right direction, the y-axis in the traveling direction, and the z-axis in the vertical direction is set with reference to the measurement device 10 (sensor 11). The left side of the x-axis is positive, the rear side of the y-axis is positive, and the upper side of the z-axis is positive. The direction of the axis set in the sensor 11 may be the same for the left and right feet, or may be different for the left and right feet. For example, in a case where the sensors 11 produced with the same specifications are arranged in the left and right shoes 100, the vertical directions (directions in the Z-axis direction) of the sensors 11 arranged in the left and right shoes 100 are the same. In this case, the three axes of the local coordinate system set in the sensor data derived from the left foot and the three axes of the local coordinate system set in the sensor data derived from the right foot are the same on the left and right. In a case where the sensors 11 produced with different specifications on the left and right are arranged in the shoes 100, the vertical directions (directions in the Z-axis direction) of the sensors 11 arranged on the left and right shoes 100 may be different.

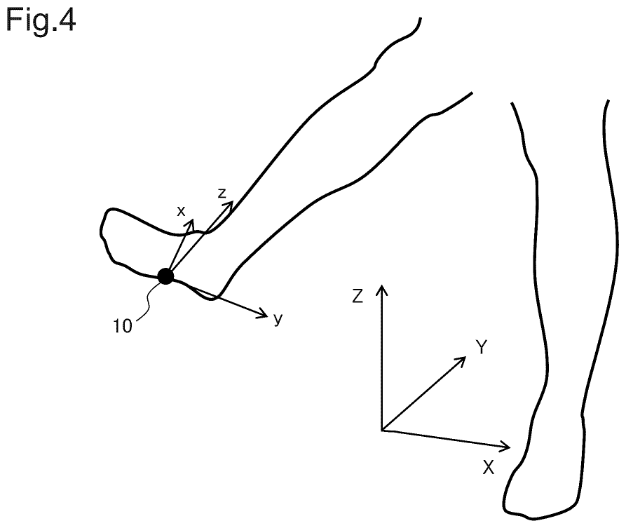

FIG. 4 is a conceptual diagram for explaining a local coordinate system (x-axis, y-axis, z-axis) set in the measurement device 10 (sensor 11) installed on the back side of the arch of the foot and a global coordinate system (X-axis, Y-axis, Z-axis) set with respect to the ground. In the global coordinate system (X-axis, Y-axis, Z-axis), in a state where the user facing the traveling direction is upright, the lateral direction of the user is set to the X-axis direction (the leftward direction is positive), the direction of the back surface of the user is set to the Y-axis direction (the rearward direction is positive), and the gravity direction is set to the Z-axis direction (the vertically upward direction is positive). The example of FIG. 4 conceptually illustrates the relationship between the local coordinate system (x-axis, y-axis, z-axis) and the global coordinate system (X-axis, Y-axis, Z-axis), and does not accurately illustrate the relationship between the local coordinate system and the global coordinate system that varies depending on the walking of the user.

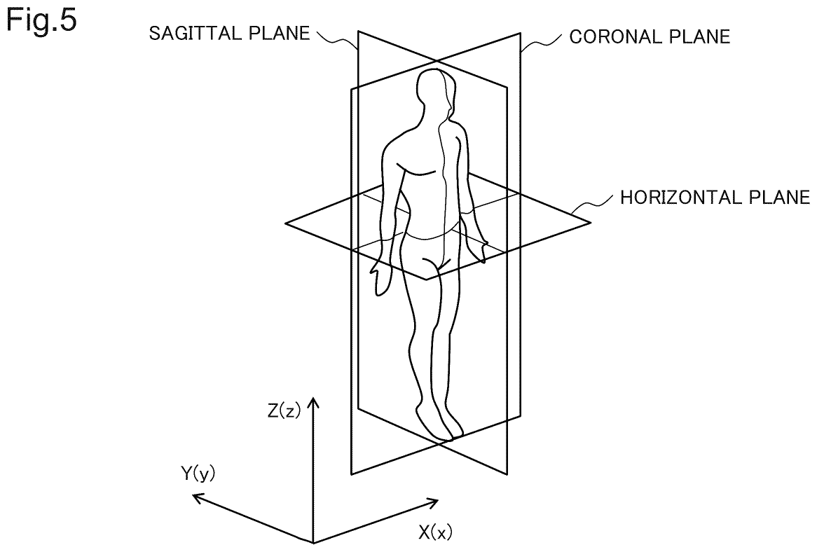

FIG. 5 is a conceptual diagram for explaining a surface (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, the global coordinate system and the local coordinate system coincide with each other in a state in which the user is upright with the center line of the foot oriented in the traveling direction. In the present example embodiment, rotation in the sagittal plane with the x-axis as the rotation axis is defined as roll, rotation in the coronal plane with the y-axis as the rotation axis is defined as pitch, and rotation in the horizontal plane with the z-axis as the rotation axis is defined as yaw. A rotation angle in the sagittal plane with the x-axis as a rotation axis is defined as a roll angle, a rotation angle in the coronal plane with the y-axis as a rotation axis is defined as a pitch angle, and a rotation angle in the horizontal plane with the z-axis as a rotation axis is defined as a yaw angle. In the following description, the x-axis, the y-axis, and the z-axis are expressed as three axes.

As illustrated in FIG. 2, the feature amount data generation unit 12 (also referred to as a feature amount data generation device) includes an acquisition unit 121, a normalization unit 122, an extraction unit 123, a generation unit 125, and a transmission unit 127. For example, the feature amount data generation unit 12 includes a microcomputer or a microcontroller that performs overall control and data processing of the measurement device 10. For example, the feature amount data generation unit 12 includes a central processing unit (CPU), a random access memory (RAM), a read only memory (ROM), a flash memory, and the like. The feature amount data generation unit 12 controls the acceleration sensor 111 and the angular velocity sensor 112 to measure the angular velocity and the acceleration. For example, the feature amount data generation unit 12 may be mounted on a mobile terminal (not illustrated) carried by a subject (user). In this case, the sensor 11 may be provided with a communication function, and the sensor data transmitted from the sensor 11 may be received by the mobile terminal on which the feature amount data generation unit 12 is mounted. When sensor data is transmitted from the sensor 11, a stable walking detection function is preferably built in the sensor 11. In this way, the sensor 11 can be configured to transmit the sensor data according to the stable walking detection. For example, stable walking can be detected when acceleration in a specific direction exceeds a predetermined threshold.

The acquisition unit 121 acquires accelerations in three axial directions from the acceleration sensor 111. The acquisition unit 121 acquires angular velocities about three axes from the angular velocity sensor 112. For example, the acquisition unit 121 performs analog-to-digital conversion (AD conversion) on the acquired physical quantities (analog data) such as an angular velocity and an acceleration. The physical quantity (analog data) measured by the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in three axial directions. The angular velocity data includes angular velocity vectors about three axes. The acceleration data and the angular velocity data are associated with acquisition time of the data. The acquisition unit 121 may add correction such as a mounting error, temperature correction, and linearity correction to the acceleration data and the angular velocity data.

The normalization unit 122 acquires sensor data from the acquisition unit 121. The normalization unit 122 extracts time-series data (also referred to as gait waveform data) for one gait cycle from the time-series data of the acceleration in the three axial directions and the angular velocity about the three axes included in the sensor data. The normalization unit 122 normalizes (also referred to as first normalization) the time of the extracted gait waveform data for one gait cycle to a gait cycle of 0 to 100% (percent). The timing such as 1% or 10% included in the 0 to 100% gait cycle is also referred to as a gait phase. The normalization unit 122 normalizes (also referred to as second normalization) the first normalized gait waveform data for one gait cycle so that the stance phase becomes 60% and the swing phase becomes 40%. The stance phase is a period in which at least a part of the back side of the foot is in contact with the ground. The swing phase is a period in which the back side of the foot is separated from the ground. When the gait waveform data is subjected to the second normalization, it is possible to reduce the influence of the deviation of the gait phase that may occur in each gait cycle.

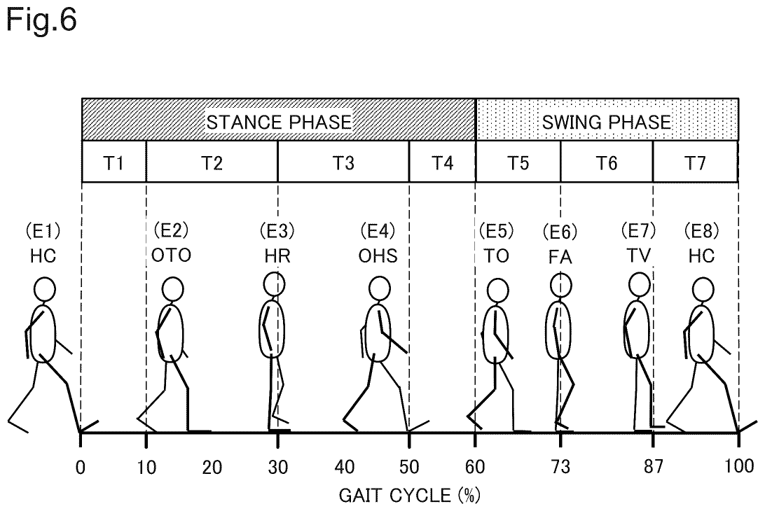

FIG. 6 is a conceptual diagram for explaining one gait cycle with the right foot as a reference. One gait cycle based on the left foot is also similar to that of the right foot. The horizontal axis of FIG. 6 is one gait cycle of the right foot with a time point at which the heel of the right foot lands on the ground as a start point and a time point at which the heel of the right foot next lands on the ground as an end point. The horizontal axis in FIG. 6 is first normalized with one gait cycle as 100%. The horizontal axis of FIG. 6 is second normalized so that the stance phase is 60% and the swing phase is 40%. The one gait cycle of one foot is roughly divided into a stance phase in which at least a part of the back side of the foot is in contact with the ground and a swing phase in which the back side of the foot is separated from the ground. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting one gait cycle, the names of these periods, and the like.

As illustrated in FIG. 6, during walking, a plurality of events (also referred to as gait events) occur. E1 represents an event (heel-contact: HC) in which the heel of the right foot touches the ground. E2 represents an event (opposite toe-off: OTO) in which the toe of the left foot is separated from the ground with the sole of the right foot in contact with the ground (OTO). E3 represents an event (heel-rise: HR) in which the heel of the right foot lifts with the sole of the right foot in contact with the ground. E4 is an event (opposite heel-strike: OHS) in which the heel of the left foot is in contact with the ground. E5 represents an event (toe-off: TO) in which the toe of the right foot is separated from the ground with the sole of the left foot in contact with the ground. E6 represents an event (foot-adjacent: FA) in which the left foot and the right foot cross with the sole of the left foot in contact with the ground. E7 represents an event (tibia-vertical: TV) that the tibia of the right foot is approximately vertical to the ground with the sole of the left foot in contact with the ground. E8 represents an event (heel-contact: HC) in which the heel of the right foot touches the ground. E8 corresponds to the end point of the gait cycle starting from E1 and corresponds to the start point of the next gait cycle. FIG. 6 is an example, and does not limit events that occur during walking or names of these events.

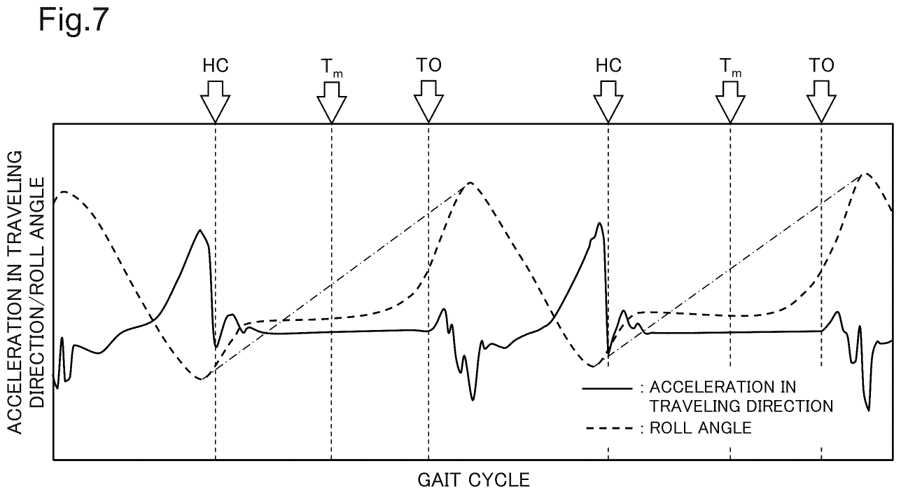

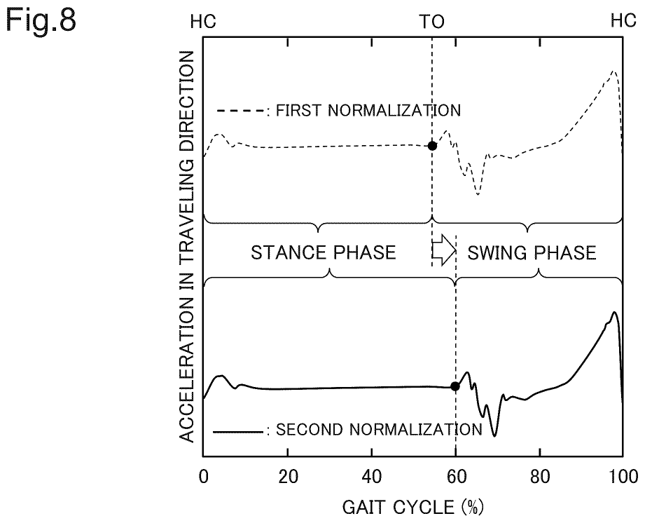

FIG. 7 is a diagram for explaining an example of detecting the heel-contact HC and the toe-off TO from the time-series data (solid line) of the traveling-direction acceleration (Y-direction acceleration). The timing of the heel-contact HC is the timing of the minimum peak immediately after the maximum peak appearing in the time-series data of the traveling-direction acceleration (Y-direction acceleration). The maximum peak serving as a mark of the timing of the heel-contact HC corresponds to the maximum peak of the gait waveform data for one gait cycle. A section between the consecutive heel-contacts HC is one gait cycle. The timing of the toe-off TO is the rising timing of the maximum peak appearing after the period of the stance phase in which a fluctuation does not appear in the time-series data of the traveling-direction acceleration (Y-direction acceleration). FIG. 8 also illustrates time-series data (broken line) of the roll angle (angular velocity about the X-axis). The timing at the midpoint between the timing of the minimum roll angle and the timing of the maximum roll angle corresponds to the timing Tₘ of the transition from the mid-stance period T2 to the terminal stance period T3. The timing Tₘ of the transition from the mid-stance period T2 to the terminal stance period T3 substantially coincides with the timing of the heel-rise HR. The parameters (also referred to as gait parameters) to be used for the estimation of the physical conditions can be obtained with reference to the timing Tₘ of the transition from the mid-stance period T2 to the terminal stance period T3.

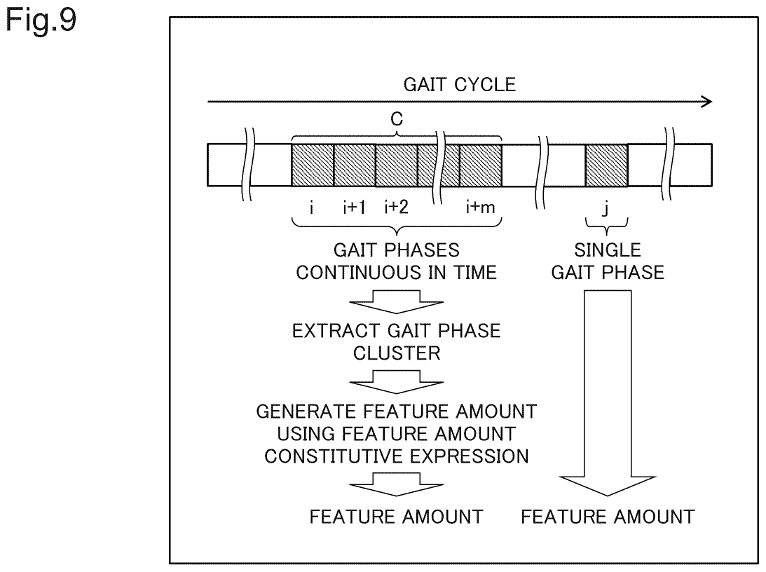

FIG. 8 is a diagram for explaining an example of normalization of gait waveform data. The normalization unit 122 detects the heel-contact HC and the toe-off TO from the time-series data of the traveling-direction acceleration (Y-direction acceleration). The normalization unit 122 extracts a section between consecutive heel-contacts HC as gait waveform data for one gait cycle. The normalization unit 122 converts the horizontal axis (time axis) of the gait waveform data for one gait cycle into a gait cycle of 0 to 100% by the first normalization. In FIG. 9, the gait waveform data after the first normalization is indicated by a broken line. In the gait waveform data (broken line) after the first normalization, the timing of the toe-off TO deviates from 60%.

In the example of FIG. 8, the normalization unit 122 normalizes a section from the heel-contact HC in which the gait phase is 0% to the toe-off TO subsequent to the heel-contact HC to 0 to 60%. The normalization unit 122 normalizes a section from the toe-off TO to the heel-contact HC in which the gait phase subsequent to the toe-off TO is 100% to 60 to 100%. As a result, the gait waveform data for one gait cycle is normalized to a section (stance phase) in which the gait cycle is 0 to 60% and a section (swing phase) in which the gait cycle is 60 to 100%. In FIG. 9, the gait waveform data after the second normalization is indicated by a solid line. In the gait waveform data (solid line) after the second normalization, the timing of the toe-off TO coincides with 60%. For example, when the times of the stance phase and the swing phase and the ratio thereof are verified, the second normalization may be omitted.

FIGS. 7 to 8 illustrate examples in which the gait waveform data for one gait cycle is extracted/normalized based on the traveling-direction acceleration (Y-direction acceleration). Regarding the acceleration/angular velocity other than the traveling-direction acceleration (Y-direction acceleration), the normalization unit 122 extracts/normalizes gait waveform data for one gait cycle according to the gait cycle of the traveling-direction acceleration (Y-direction acceleration). The normalization unit 122 may generate time-series data of angles about three axes by integrating time-series data of angular velocities about the three axes. In this case, the normalization unit 122 also extracts/normalizes the gait waveform data for one gait cycle according to the gait cycle of the traveling-direction acceleration (Y-direction acceleration) with respect to the angles about the three axes.

The normalization unit 122 may extract/normalize the gait waveform data for one gait cycle based on acceleration/angular velocity other than the traveling-direction acceleration (Y-direction acceleration) (not illustrated). For example, the normalization unit 122 may detect the heel-contact HC and the toe-off TO from the time-series data of the vertical-direction acceleration (Z-direction acceleration). The timing of the heel-contact HC is a timing of a steep minimum peak appearing in the time-series data of the vertical-direction acceleration (Z-direction acceleration). At the timing of the steep minimum peak, the value of the vertical-direction acceleration (Z-direction acceleration) becomes substantially zero. The minimum peak serving as a mark of the timing of the heel-contact HC corresponds to the minimum peak of the gait waveform data for one gait cycle. A section between the consecutive heel-contacts HC is one gait cycle. The timing of the toe-off TO is a timing of an inflection point in the middle of gradually increasing after the time-series data of the vertical-direction acceleration (Z-direction acceleration) passes through a section with a small fluctuation after the maximum peak immediately after the heel-contact HC. The normalization unit 122 may extract/normalize the gait waveform data for one gait cycle based on both the traveling-direction acceleration (Y-direction acceleration) and the vertical-direction acceleration (Z-direction acceleration). The normalization unit 122 may extract/normalize the gait waveform data for one gait cycle based on acceleration, angular velocity, angle, and the like other than the traveling-direction acceleration (Y-direction acceleration) and the vertical-direction acceleration (Z-direction acceleration).

The extraction unit 123 acquires gait waveform data for one gait cycle normalized by the normalization unit 122. The extraction unit 123 extracts a feature amount to be used for estimation of the harmonic index from the gait waveform data for one gait cycle. The extraction unit 123 extracts a feature amount (also referred to as a cluster feature amount) for each gait phase cluster from the gait phase clusters obtained by integrating temporally consecutive gait phases based on a preset condition. The gait phase cluster includes at least one gait phase. The gait phase cluster may also be composed of a single gait phase. The gait waveform data and the gait phase from which the feature amount to be used for estimation of the harmonic index is extracted will be described later.

FIG. 9 is a conceptual diagram for explaining extraction of a feature amount for estimating a harmonic index from gait waveform data for one gait cycle. For example, the extraction unit 123 extracts temporally consecutive gait phases i to i+m as the gait phase cluster C (i and m are natural numbers). In the present example embodiment, an example in which the gait phase cluster C to be used for estimation of the harmonic index is selected by correlation analysis using statistic parametric mapping will be described. For example, the gait phase cluster C may be selected by Pearson's correlation analysis.

In the example of FIG. 9, the gait phase cluster C includes m gait phases (components). That is, the number of gait phases (components) (also referred to as the number of components) constituting the gait phase cluster C is m. FIG. 9 illustrates an example in which the number of gait phases has an integer value, but the number of gait phases may be subdivided into decimal places. When the number of gait phases is subdivided into decimal places, the number of components of the gait phase cluster C is a number corresponding to the number of data points in the section of the gait phase cluster. The extraction unit 123 extracts a feature amount from each of the gait phases i to i+m. In a case where the gait phase cluster C is composed of a single gait phase j, the extraction unit 123 extracts a feature amount from the single gait phase j (j is a natural number).

The generation unit 125 applies the feature amount constitutive expression to the feature amount extracted from each of the gait phases constituting the gait phase cluster to generate the feature amount (cluster feature amount) of the gait phase cluster. The feature amount constitutive expression is a preset calculation formula for generating the feature amount of the gait phase cluster. For example, the feature amount constitutive expression is a calculation formula related to four arithmetic operations. For example, the cluster feature amount calculated using the feature amount constitutive equation is an integral average value, an arithmetic average value, an inclination, a variation, or the like of the feature amount in each gait phase included in the gait phase cluster. For example, the generation unit 125 applies a calculation formula for calculating the inclination or variation of the feature amount extracted from each of the gait phases constituting the gait phase cluster as the feature amount constitutive formula. For example, in a case where the gait phase cluster is composed of a single gait phase, it is not possible to calculate the inclination or variation, and thus, it is sufficient to use a feature amount constituent equation for calculating an integral average value, an arithmetic average value, or the like. For example, in a case where the gait phase cluster is composed of a single gait phase, the feature amount extracted from the gait phase may be set as the cluster feature amount.

The generation unit 125 calculates parameters (also referred to as gait parameters) related to the gait. The generation unit 125 calculates the gait parameters using the feature amount derived from the gait waveform data. The gait parameters include features to be used for estimation of physical conditions. The estimation system 1 may be configured to calculate the gait parameters on the side of the harmonic index estimation device 13. Hereinafter, examples of the gait parameters calculated by the generation unit 125 will be listed. The following gait parameters are merely examples, and do not cover all parameters including the features of the gait. In the present example embodiment, among the following gait parameters, those having a high correlation in estimation of the harmonic index are selected. Details of the method for calculating the gait parameters will be omitted.

Examples of the gait parameters include a stride, a walking pitch, a walking speed, a contact angle, a take-off angle, an outward turning distance (diversion amount), and a toe direction (inward/outward turning). The stride is a distance between the toes of both feet in a state in which one step is taken with one of the left and right feet and the toes land on the ground. The walking pitch is the number of steps within a predetermined time, and is used for calculating the walking speed. The walking speed is a moving speed in one gait cycle. The walking speed may be a value averaged in a plurality of gait cycles. The contact angle is an angle (posture angle) of the sole with respect to the ground in a state where the heel is in contact with the ground. The contact angle is an angle (posture angle) of the sole with respect to the ground in a state where the toes are in contact with the ground. The outward turning distance is a distance between a foot and a straight line indicating a moving route at a timing when the foot is farthest from the moving route of one foot in one gait cycle. The toe direction is an angle between a straight line indicating a movement route of one foot in one gait cycle and a center line of the foot in a landed state.

Examples of the gait parameters include a roll angle, a foot lift height, a maximum angular velocity in a plantarflexion direction, a maximum angular velocity in a dorsiflexion direction, a maximum speed, a maximum acceleration of a foot during the swing phase, and a cadence. For example, the roll angle at the heel-contact or the toe-off is used as the gait parameters. The foot lift height corresponds to the height of the foot in the vertical direction. For example, the maximum angular velocity in the plantarflexion direction and the maximum angular velocity in the dorsiflexion direction during the swing phase are used as the gait parameters. For example, the maximum speed during the swing phase is used as the gait parameters. The maximum acceleration of the foot during the swing phase is the maximum value of the vertical-direction acceleration of the foot during the swing phase, and relates to the rise of the waist according to the interlocking of the movement of the foot and the waist. The cadence corresponds to the number of steps in 60 seconds.

Examples of the gait parameters include a stance time, a swing time, a double support time (DST), a load time, a plantar contact time, and a kicking time. The stance time is a time corresponding to the period of the stance phase. The swing time is a time corresponding to the period of the swing phase. The DST corresponds to a double-leg support period during walking. The DST includes a DST1 corresponding to a double-leg support period after the heel-contact and a DST2 corresponding to a double-leg support period immediately before kicking. The load time is a time during which a load is applied to the sole of the foot. The load time corresponds to a time from heel-contact to plantar contact. The plantar contact time is a time during which the main surface of the plantar surface is in contact with the ground. The plantar contact time corresponds to the time from the plantar contact to the heel-off. The kicking time is a time from application of a load to the main surface of the sole of the foot to kicking of the foot. The kicking time corresponds to the time from the plantar contact to the toe-off.

The transmission unit 127 outputs the feature amount data including the cluster feature amounts and the gait parameters generated by the generation unit 125. The cluster feature amounts and the gait parameters are also referred to as first feature amounts. The transmission unit 127 transmits the feature amount data to the harmonic index estimation device 13. For example, the transmission unit 127 transmits the feature amount data to the harmonic index estimation device 13 via wireless communication. For example, the transmission unit 127 is configured to transmit the feature amount data to the harmonic index estimation device 13 via a wireless communication function (not illustrated) conforming to a standard such as Bluetooth (registered trademark) or WiFi (registered trademark). The communication function of the transmission unit 127 may conform to a standard other than Bluetooth (registered trademark) or WiFi (registered trademark).

### Harmonic Index Estimation Device

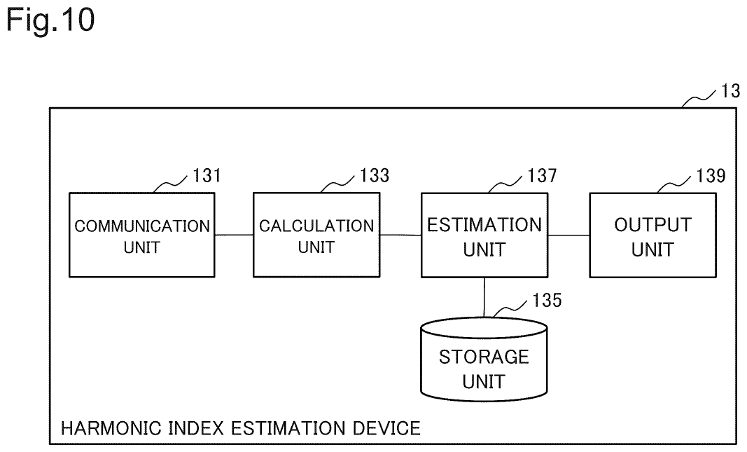

FIG. 10 is a block diagram illustrating an example of a configuration of the harmonic index estimation device 13. The harmonic index estimation device 13 includes a communication unit 131, a calculation unit 133, a storage unit 135, an estimation unit 137, and an output unit 139.

The communication unit 131 acquires the feature amount data from the measurement device 10. The communication unit 131 outputs the received data to the calculation unit 133. The communication unit 131 receives the feature amount data from the measurement device 10 via wireless communication. For example, the communication unit 131 is configured to receive the feature amount data from the measurement device 10 via a wireless communication function (not illustrated) conforming to a standard such as Bluetooth (registered trademark) or WiFi (registered trademark). The communication function of the communication unit 131 may conform to a standard other than Bluetooth (registered trademark) or WiFi (registered trademark). The communication unit 131 may receive the feature amount data from the measurement device 10 via a wire such as a cable.

The calculation unit 133 acquires the feature amount data. The calculation unit 133 calculates input data to be used for estimation of the harmonic index using the cluster feature amounts and the gait parameters included in the acquired feature amount data. The calculation unit 133 calculates the average value and the absolute value of the difference with respect to the first feature amounts (cluster feature amounts/gait parameters) for both feet used for estimation of the harmonic index. Hereinafter, the absolute value of the difference is also referred to as a difference. The average value and the difference of the first feature amounts for both feet calculated by the calculation unit 133 are also referred to as second feature amounts. The second feature amounts are used for estimation of the harmonic index. Instead of the average value or the difference of the first feature amounts included in the feature amount data generated by the measurement device 10, the first feature amounts may be directly used for estimation of the harmonic index. In this case, the calculation unit 133 can be omitted.

The storage unit 135 stores an estimation model for estimating the harmonic index using the feature amount data extracted from the gait waveform data. The estimation model outputs the estimation result related to the harmonic index according to the input of the input data calculated by the calculation unit 133. The storage unit 135 stores estimation models learned for a plurality of subjects. In a case where the attribute of the subject is used for estimation, the storage unit 135 stores the attribute of the subject. For example, the attribute of the subject includes gender, age, weight, height, and the like of the subject. The harmonic index estimation device 13 estimates the harmonic indices in the three directions of the traveling direction, the left-right direction, and the vertical direction. The attribute of the subject varies depending on the direction of the harmonic index to be estimated.

The estimation model is stored in the storage unit 135 at the time of factory shipment of a product, calibration before the user uses the estimation system, or the like. For example, the estimation system 1 may be configured to use an estimation model stored in a storage device such as an external server. In that case, the estimation system 1 may be configured such that the estimation model is used via an interface (not illustrated) connected to the storage device.

The estimation unit 137 acquires, from the calculation unit 133, input data to be used for estimation of the harmonic index. When the feature amount data generated by the measurement device 10 is used as it is, the estimation unit 137 acquires the feature amount data as input data. In a case where the attribute of the subject is used for estimation, the estimation unit 137 acquires the attribute of the subject from the storage unit 135.

The estimation unit 137 estimates the harmonic index using the acquired input data. In the present example embodiment, an example of estimating HR of the waist in one gait cycle will be described. The HR of the waist corresponds to a ratio between even-numbered frequency components and odd-numbered frequency components or a ratio between odd-numbered frequency components and even-numbered frequency components obtained by performing Fourier transform on time-series data of acceleration of the waist in one gait cycle. The HR in the vertical direction and the traveling direction corresponds to a ratio between even-numbered frequency components and odd-numbered frequency components. The HR in the left-right direction corresponds to a ratio between odd-numbered frequency components and even-numbered frequency components.

The estimation unit 137 inputs input data to the estimation model stored in the storage unit 135. The estimation unit 137 outputs the estimation result of the harmonic index output from the estimation model. In a case where an estimation model stored in an external storage device constructed in a cloud, a server, or the like is used, the estimation unit 137 is configured to use the estimation model via an interface (not illustrated) connected to the storage device.

The harmonic index is an index related to smoothness of movement of the waist and harmonicity of walking. The estimation unit 137 estimates the harmonic indices in the three directions of the left-right direction, the traveling direction, and the vertical direction. The harmonic index of the traveling direction is associated to the smoothness of the movement of the waist in the front-back direction (in the sagittal plane) about the pelvis. The harmonic index in the left-right direction is associated to smoothness of movement of the waist in the vertical direction (in the coronal plane) about the pelvis. The harmonic index in the vertical direction is associated to the smoothness of the movement of the waist in the rotation (in the horizontal plane) of the body about the pelvis. Using the harmonic index, the smoothness of the movement of the subject and the harmonicity of walking, which cannot be grasped only by the movement of the foot, can be grasped.

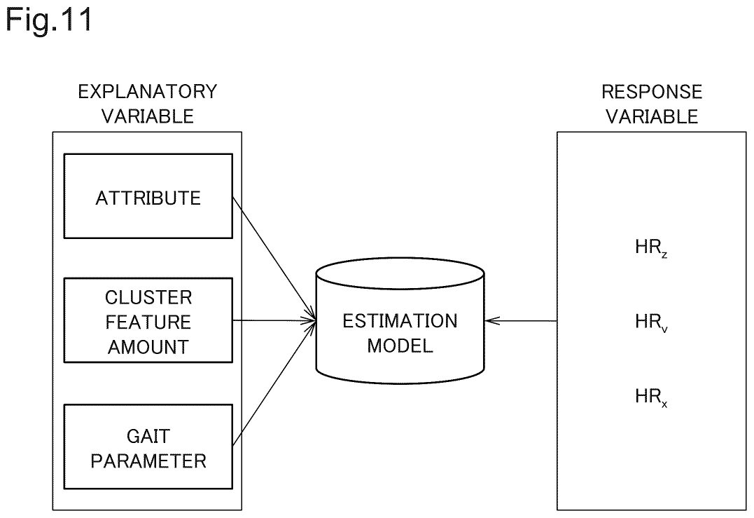

FIG. 11 is a conceptual diagram for explaining an example of learning of an estimation model used for estimation of a harmonic index. For learning of the estimation model, explanatory variables and response variables related to a plurality of subjects are used. As the explanatory variable, the second feature amounts generated based on the attribute of the subject, the cluster feature amount generated according to the walking of the subject, and the gait parameters are used. As the explanatory variable, the first feature amounts including the cluster feature amounts and the gait parameters generated according to the walking of the subject may be used. As the response variable, a measured value of the harmonic index of the spatial acceleration related to the waist is used. For example, the measured value of the harmonic index is measured by the IMU attached to the waist. The harmonic index HR_{z} in the vertical direction, the harmonic index HR_{y} in the traveling direction, and the harmonic index HR_{z} in the left-right direction are used as response variables. For example, a harmonic index in at least one of the vertical direction, the traveling direction, and the left-right direction may be used as the response variable. For example, the harmonic index in the vertical direction can be calculated by performing Fourier transform on the vertical-direction acceleration and calculating the ratio of the power sum of the even components and the power sum of the odd components included in the frequency components. For example, the harmonic index in the traveling direction can be calculated by performing Fourier transform on the traveling-direction acceleration and calculating the ratio of the power sum of the even components and the power sum of the odd components included in the frequency components. The harmonic index in the left-right direction can be calculated by performing Fourier transform on the horizontal-direction acceleration and calculating the ratio of the power sum of the odd components and the power sum of the even components included in the frequency components. For example, the estimation model is a multiple regression model constructed using the feature amount selected by the Leave-one-subject-out LASSO method.

For example, the estimation model is constructed by learning using a linear regression algorithm. For example, the estimation model is constructed by learning using a support vector machine (SVM) algorithm. For example, the estimation model is constructed by learning using a Gaussian Process Regression (GPR) algorithm. For example, the estimation model is constructed by learning using a random forest (RF) algorithm. The estimation model may be constructed by unsupervised learning that classifies subjects who are generation sources of the feature amount data according to the feature amount data. The algorithm used for learning the estimation model is not particularly limited.

The estimation model may be constructed by learning using gait waveform data (sensor data) for one gait cycle as an explanatory variable. For example, the estimation model is constructed by supervised learning in which the gait waveform data of the acceleration in the three axial directions, the angular velocity about the three axes, and the angles (posture angles) about the three axes is used as explanatory variables and the harmonic index to be estimated is used as an objective variable.

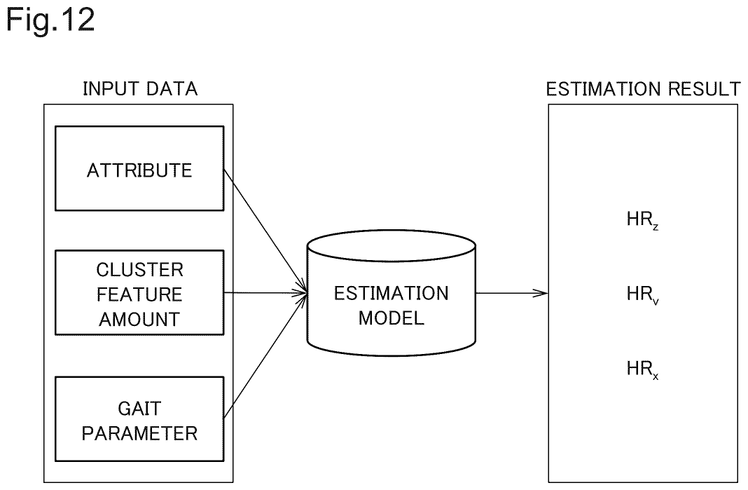

FIG. 12 is a conceptual diagram illustrating an example of estimating a harmonic index of a subject (user) using an estimation model. The estimation model is a model constructed in advance by learning illustrated in FIG. 11. In the example of FIG. 12, the second feature amount generated based on the attribute of the subject and the first feature amounts (cluster feature amounts/gait parameters) generated according to the walking of the subject is used as the input data. In the case of the estimation model generated using the first feature amounts including the cluster feature amounts and the gait parameters, the cluster feature amounts and the gait parameters are used as the input data. The estimation model outputs the harmonic index according to the input of the input data. In the example of FIG. 12, the harmonic index HR_{z} in the vertical direction, the harmonic index HR_{y} in the traveling direction, and the harmonic index HRₓ in the left-right direction are output as the estimation results. For example, the estimation model may be configured such that at least one of the harmonic index HR_{z} in the vertical direction, the harmonic index HR_{y} in the traveling direction, and the harmonic index HRₓ in the left-right direction is output as an estimation result.

The output unit 139 outputs the estimation result of the harmonic index by the estimation unit 137. For example, the output unit 139 displays the estimation result of the harmonic index on the screen of the mobile terminal of the subject (user). For example, the output unit 139 outputs the estimation result to an external system or the like that uses the estimation result. There is no particular limitation on the use of the information related to the harmonic index output from the harmonic index estimation device 13.

For example, the harmonic index estimation device 13 is connected to an external system or the like built in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the harmonic index estimation device 13 is connected to a mobile terminal via a wire such as a cable. For example, the harmonic index estimation device 13 is connected to a mobile terminal via wireless communication. For example, the harmonic index estimation device 13 is connected to a mobile terminal via a wireless communication function (not illustrated) conforming to a standard such as Bluetooth (registered trademark) or WiFi (registered trademark). The communication function of the harmonic index estimation device 13 may conform to a standard other than Bluetooth (registered trademark) or WiFi (registered trademark). The estimation result of the harmonic index may be used by an application installed in the mobile terminal. In that case, the mobile terminal executes processing using the estimation result by application software or the like installed in the mobile terminal.

### Operation

Next, an operation of the estimation system 1 will be described with reference to the drawings. Here, the measurement device 10 and the harmonic index estimation device 13 included in the estimation system 1 will be individually described. Regarding the measurement device 10, the operation of the feature amount data generation unit 12 included in the measurement device 10 will be described.

### Measurement Device

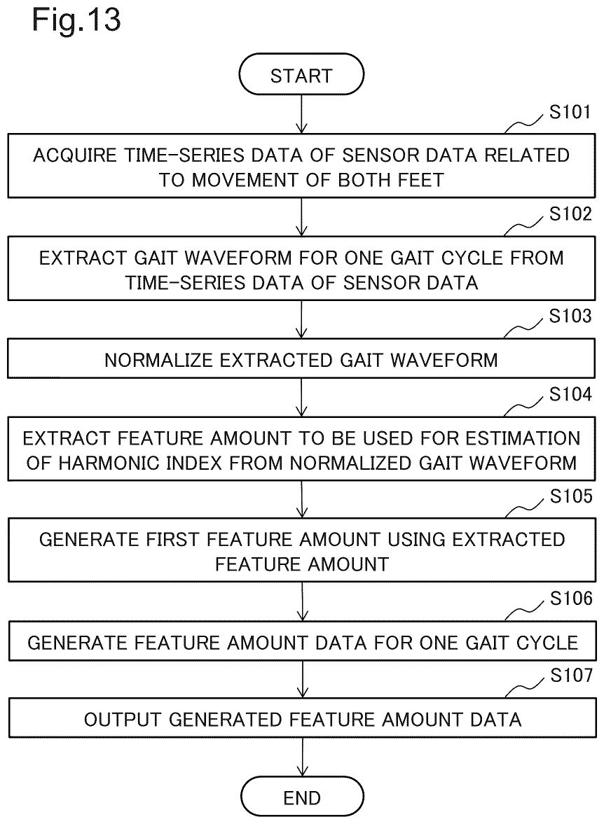

FIG. 13 is a flowchart for explaining the operation of the feature amount data generation unit 12 included in the measurement device 10. In the description according to the flowchart of FIG. 13, the feature amount data generation unit 12 will be described as the subject of an operation.

In FIG. 13, first, the feature amount data generation unit 12 acquires time-series data of sensor data related to the movement of both feet (step S101).

Next, the feature amount data generation unit 12 extracts gait waveform data for one gait cycle from the time-series data of the sensor data (step S102). The feature amount data generation unit 12 detects heel-contact and toe-off from the time-series data of the sensor data. The feature amount data generation unit 12 extracts time-series data in a section between consecutive heel-contacts as gait waveform data for one gait cycle.

Next, the feature amount data generation unit 12 normalizes the extracted gait waveform data for one gait cycle (step S103). The feature amount data generation unit 12 normalizes the gait waveform data for one gait cycle to a gait cycle of 0 to 100% (first normalization). The feature amount data generation unit 12 normalizes the ratio of the stance phase to the swing phase in the first normalized gait waveform data for one gait cycle to 60:40 (second normalization).

Next, the feature amount data generation unit 12 extracts a feature amount from the gait phase used for estimation of the harmonic index with respect to the normalized gait waveform (step S104). The feature amount data generation unit 12 extracts a feature amount to be used for estimation of the harmonic index.

Next, the feature amount data generation unit 12 generates a first feature amount using the extracted feature amount (step S105). The feature amount data generation unit 12 generates a first feature amount including cluster feature amounts and gait parameters according to the harmonic index to be estimated.

Next, the feature amount data generation unit 12 integrates the first feature amounts for one gait cycle to generate feature amount data for one gait cycle (step S106).

Next, the feature amount data generation unit 12 outputs the generated feature amount data to the harmonic index estimation device 13 (step S107).

### Harmonic Index Estimation Device

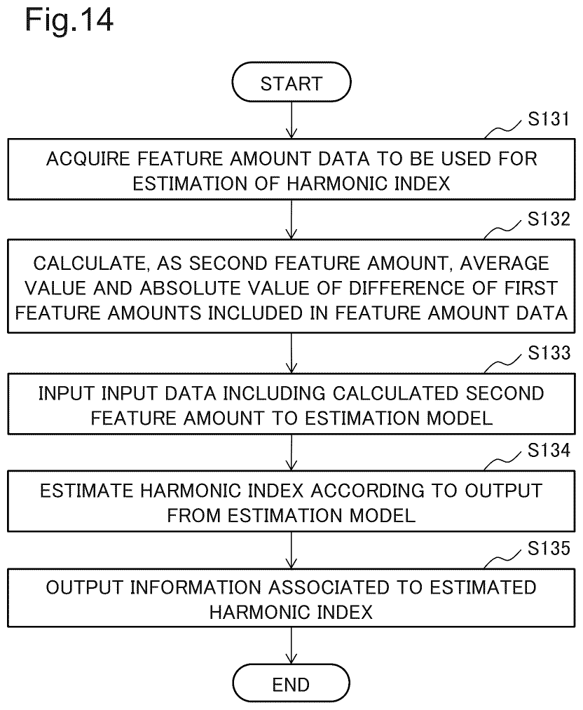

FIG. 14 is a flowchart for explaining an operation of the harmonic index estimation device 13. In the description according to the flowchart of FIG. 14, the harmonic index estimation device 13 will be described as the subject of an operation.

In FIG. 14, first, the harmonic index estimation device 13 acquires feature amount data to be used for estimation of the harmonic index from the measurement device 10 (step S131).

Next, the harmonic index estimation device 13 calculates, as the second feature amount, the average value and the absolute value of the difference of the first feature amounts included in the acquired feature amount data (step S132).

Next, the harmonic index estimation device 13 inputs input data including the calculated second feature amount to an estimation model for estimating a harmonic index (step S133).

Next, the harmonic index estimation device 13 estimates the user's harmonic index according to the output (estimation value) from the estimation model (step S134).

Next, the harmonic index estimation device 13 outputs information corresponding to the estimated harmonic index (step S135). For example, the harmonic index is output to a terminal device (not illustrated) carried by the user. For example, information corresponding to the harmonic index is output to a system that executes processing using the information.

### Application Example

Next, an application example according to the present example embodiment will be described with reference to the drawings. In the following application example, an example in which the function of the harmonic index estimation device 13 installed in the mobile terminal carried by the user estimates the information on the harmonic index using the feature amount data measured by the measurement device 10 arranged in the shoe will be described.

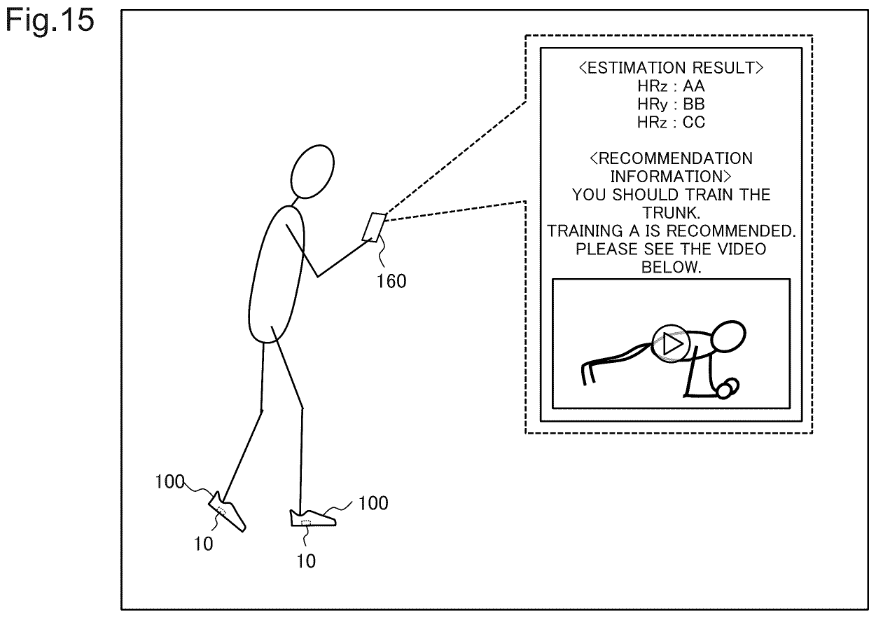

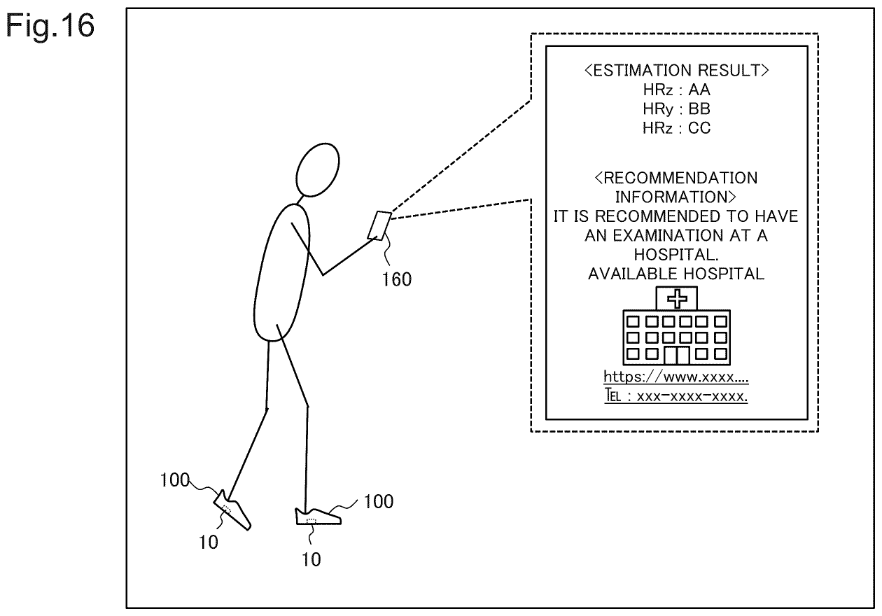

FIGS. 15 to 16 are conceptual diagrams illustrating an example in which the estimation result by the harmonic index estimation device 13 is displayed on the screen of a mobile terminal 160 carried by the user walking while wearing the shoe 100 on which the measurement device 10 is disposed. In the examples of FIGS. 15 to 16, the information corresponding to the estimation result of the harmonic index using the feature amount data corresponding to the sensor data measured while the user is walking is displayed on the screen of the mobile terminal 160.

In the example of FIG. 15, the estimation results of the harmonic indices in the three directions of the traveling direction, the left-right direction, and the vertical direction are displayed on the display unit of the mobile terminal 160. In the example of FIG. 15, according to the estimation value of the harmonic index, recommendation information corresponding to the estimation result of the harmonic index of “You should train the trunk.” is displayed on the display unit of the mobile terminal 160. In the example of FIG. 15, according to the estimation value of the harmonic index, recommendation information corresponding to the estimation result of “Training A is recommended. Please see the video below.” is displayed on the display unit of the mobile terminal 160. The user who has confirmed the information displayed on the display unit of the mobile terminal 160 can practice training for training the trunk by exercising with reference to the video of the training A according to the recommendation information.

In the example of FIG. 16, the estimation results of the harmonic indices in the three directions of the traveling direction, the left-right direction, and the vertical direction are displayed on the display unit of the mobile terminal 160. In the example of FIG. 16, recommendation information of “It is recommended to have an examination at a hospital.” is displayed on the display unit of the mobile terminal 160 according to the estimation value of the harmonic index. For example, a link destination or a telephone number to a hospital site where the user can be examined may be displayed on the screen of the mobile terminal 160. The user who has confirmed the information displayed on the display unit of the mobile terminal 160 can appropriately receive an examination of a disease related to the knee by visiting a hospital according to the recommendation information.

As described above, the estimation system of the present example embodiment includes the measurement device and the harmonic index estimation device. The measurement device is installed on the footwear of the subject who is an estimation target of the harmonic index that is the index related to the movement of the waist. The measurement device includes a sensor and a feature amount data generation unit. The sensor measures a spatial acceleration and a spatial angular velocity. The sensor generates sensor data related to the movement of the foot using the measured spatial acceleration and spatial angular velocity. The sensor outputs the generated sensor data. The feature amount data generation unit acquires time-series data of sensor data including features of a gait. The feature amount data generation unit extracts gait waveform data for one gait cycle from the time-series data of the sensor data. The feature amount data generation unit normalizes the extracted gait waveform data. The feature amount data generation unit extracts, from the normalized gait waveform data, a feature amount to be used for estimation of the harmonic index from a gait phase cluster including at least one temporally consecutive gait phase. The feature amount data generation unit generates feature amount data including the extracted feature amount. The feature amount data generation unit outputs the generated feature amount data to the harmonic index estimation device.

The harmonic index estimation device includes a communication unit, a storage unit, an estimation unit, and an output unit. The communication unit acquires the feature amount data including the first feature amount extracted from the gait waveforms of the spatial acceleration and the spatial angular velocity included in the sensor data related to the movement of the foot of the subject and used for estimation of the harmonic index related to the smoothness of the movement of the waist. The first feature amount includes at least one of gait parameters and a cluster feature amount for each gait phase cluster.

The storage unit stores an estimation model that outputs an estimation value related to the harmonic index according to an input of the first feature amount included in the feature amount data. The estimation unit inputs the first feature amount included in the acquired feature amount data to the estimation model, and estimates the harmonic index of the subject according to the estimation value related to the harmonic index output from the estimation model. The output unit outputs information corresponding to the harmonic index of the subject.

In the present example embodiment, the harmonic index related to smoothness of the movement of the waist of the subject is estimated using the first feature amount extracted from the sensor data related to the movement of the foot of the subject. Therefore, according to the present example embodiment, it is possible to easily estimate the harmonic index related to the smoothness of the movement of the waist with high accuracy in daily life.

A harmonic index estimation device according to one aspect of the present example embodiment includes a calculation unit (first calculation unit). The calculation unit calculates, as the second feature amount, an average value and a difference of the first feature amounts to be used for estimation of the harmonic index among the first feature amounts for both feet of the subject. The storage unit stores an estimation model that outputs an estimation value related to the harmonic index according to the input of the second feature amount. The estimation unit inputs the calculated second feature amount to the estimation model, and estimates the harmonic index of the subject according to the estimation value related to the harmonic index output from the estimation model. According to the present aspect, the harmonic index can be estimated with higher accuracy using the second feature amount generated using the first feature amounts for both feet.

In one aspect of the present example embodiment, the storage unit stores an estimation model that outputs an estimation value related to the harmonic index according to the input of the attribute of the subject and the second feature amount. The estimation unit inputs the attribute of the subject and the second feature amount to the estimation model, and estimates the harmonic index of the subject according to the estimation value related to the harmonic index output from the estimation model. According to the present aspect, the harmonic index can be estimated with higher accuracy using the attribute of the subject.

In one aspect of the present example embodiment, the storage unit stores an estimation model that outputs an estimation value related to the harmonic index according to an input of the first feature amount included in the feature amount data. The estimation model outputs at least one harmonic indices in the three directions of a traveling direction, a left-right direction, and a vertical direction in one gait cycle as an estimation value related to the harmonic index. The estimation unit inputs the first feature amount included in the acquired feature amount data to the estimation model, and estimates the harmonic index of the subject according to the harmonic index output from the estimation model. The estimation model outputs at least one harmonic indices in the three directions of the traveling direction, the left-right direction, and the vertical direction. According to the present aspect, the harmonic index can be estimated with higher accuracy according to the harmonic indices in the three directions of the traveling direction, the left-right direction, and the vertical direction.

In one aspect of the present example embodiment, the harmonic index estimation device is mounted on a terminal device having a screen visually recognizable by a subject. The harmonic index estimation device displays, on the screen of the terminal device, information on the harmonic index estimated according to the movement of the foot of the subject. According to the present aspect, the information on the harmonic index estimated for the subject can be accurately presented to the subject.

The harmonic index HR is an index of physical conditions or health conditions. A person with a sufficiently large harmonic index HR is healthy. A person with a small harmonic index HR may have a risk of fall or progressed low back pain. Therefore, the harmonic index HR can be used for determining the risk of fall or the degree of progress of low back pain. For example, regarding the degree of progress of the symptom to be detected, a threshold related to the harmonic index HR is set based on verification for a plurality of subjects. In this way, the degree of progress of the symptom can be estimated according to the estimation result of the harmonic index HR.

### Second Example Embodiment

Next, an estimation system according to a second example embodiment will be described with reference to the drawings. The estimation system of the present example embodiment estimates odd components and even components included in frequency components obtained by performing Fourier transform on time-series data of a spatial acceleration using different estimation models. The estimation system of the present example embodiment estimates the harmonic index using the estimated odd components and even components.

### Configuration

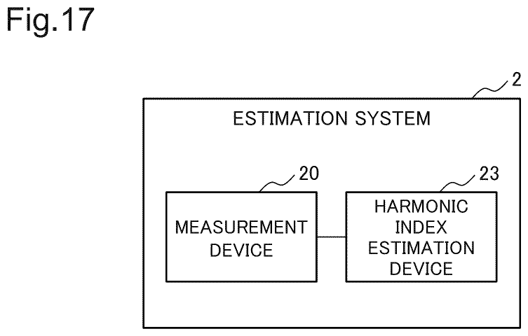

FIG. 17 is a block diagram illustrating an example of a configuration of an estimation system 2 according to the present example embodiment. The estimation system 2 includes a measurement device 20 and a harmonic index estimation device 23. In the present example embodiment, an example in which the measurement device 20 and the harmonic index estimation device 23 are configured as separate pieces of hardware will be described. For example, the measurement device 20 is installed on footwear of a subject (user) who is an estimation target of the harmonic index. For example, the function of the harmonic index estimation device 23 is installed in a mobile terminal carried by a subject (user). The measurement device 20 has the same configuration as the measurement device 10 of the first example embodiment. Hereinafter, the description of the measurement device 20 will be omitted, and the configuration of the harmonic index estimation device 23 will be described.

### Harmonic Index Estimation Device

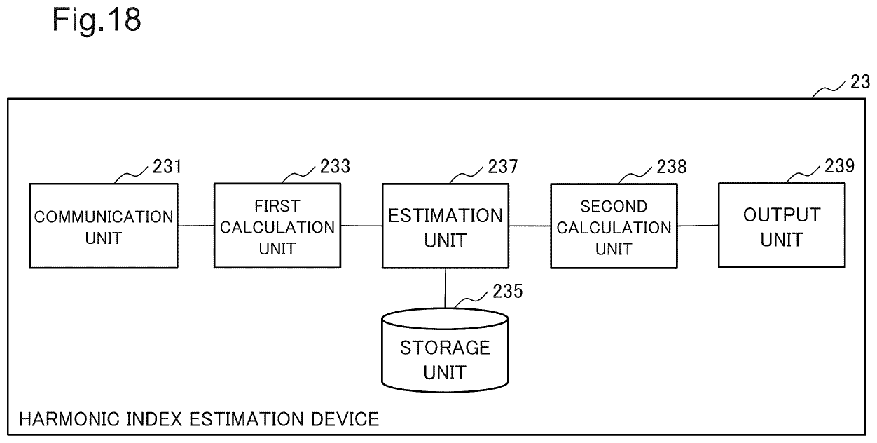

FIG. 18 is a block diagram illustrating an example of a configuration of the harmonic index estimation device 23. The harmonic index estimation device 23 includes a communication unit 231, a first calculation unit 233, a storage unit 235, an estimation unit 237, a second calculation unit 238, and an output unit 239.

The communication unit 231 has the same configuration as the communication unit 131 of the first example embodiment. The communication unit 231 acquires the feature amount data from the measurement device 20. The communication unit 231 outputs the received data to the first calculation unit 233.

The first calculation unit 233 is similar to the calculation unit 133 of the first example embodiment. The first calculation unit 233 acquires the feature amount data. The first calculation unit 233 calculates input data to be used for estimation of frequency components (odd components/even components) used for calculation of the harmonic index using the cluster feature amounts and the gait parameters included in the acquired feature amount data. The first calculation unit 233 calculates an average value of the first feature amounts for both feet used for estimation of the harmonic index. The first calculation unit 233 calculates the absolute value of the difference of the first feature amounts for both feet used for estimation of the harmonic index. The first calculation unit 233 calculates an average value of the gait parameters of both feet used for estimation of the harmonic index. The first calculation unit 233 calculates the absolute value of the difference of the gait parameters for both feet used for estimation of the harmonic index. Hereinafter, the absolute value of the difference is also referred to as a difference. The average value or difference of the first feature amounts/gait parameters for both feet calculated by the first calculation unit 233 is the second feature amount. The second feature amount is used for estimation of frequency components (odd components/even components). Instead of the average value or difference of the first feature amounts or the gait parameters included in the feature amount data generated by the measurement device 20, the first feature amounts or the gait parameters may be directly used for estimation of the frequency components (odd components/even components). In that case, the first calculation unit 233 may be omitted.

The storage unit 235 stores an estimation model for estimating frequency components (odd components/even components) used for calculation of the harmonic index. The storage unit 235 stores two estimation models for estimating the odd components and the even components included in the frequency components. In the present example embodiment, an example of estimating the logarithmically transformed odd components/even components will be described. A model for estimating odd components included in frequency components is referred to as a first estimation model. The first estimation model outputs an estimation result related to the logarithmically transformed odd components according to the input of the input data calculated by the first calculation unit 233. A model for estimating even components included in frequency components is referred to as a second estimation model. The second estimation model outputs an estimation result related to the logarithmically transformed even components according to the input of the input data calculated by the first calculation unit 233. The storage unit 235 stores estimation models learned for a plurality of subjects. In a case where the attribute of the subject is used for estimation, the storage unit 235 stores the attribute of the subject. For example, the attribute of the subject includes gender, age, weight, height, and the like of the subject. The harmonic index estimation device 23 estimates the logarithmically transformed odd components/even components used for calculation of the harmonic index in each of three directions of the vertical direction, the traveling direction, and the left-right direction. The attribute of the subject varies depending on the direction of the harmonic index to be estimated.

The estimation model is stored in the storage unit 235 at the time of factory shipment of a product, calibration before the user uses the estimation system, or the like. For example, the estimation system 1 may be configured to use an estimation model stored in a storage device such as an external server. In that case, the estimation system 2 may be configured such that the estimation model is used via an interface (not illustrated) connected to the storage device.

The estimation unit 237 acquires, from the first calculation unit 233, input data to be used for estimation of frequency components (odd components/even components) used for calculation of the harmonic index. When the feature amount data generated by the measurement device 20 is used as it is, the estimation unit 237 acquires the feature amount data as input data. In a case where the attribute of the subject is used for estimation, the estimation unit 237 acquires the attribute of the subject from the storage unit 235.

The estimation unit 237 estimates frequency components (odd components/even components) used for calculation of the harmonic index using the acquired input data. In the present example embodiment, an example of estimating the logarithmically transformed odd components and even components will be described. The harmonic index in the vertical direction and the traveling direction is a ratio between odd components and even components. The harmonic index in the left-right direction is a ratio between even components and odd components.

The estimation unit 237 inputs input data for estimating the logarithmically transformed odd components to the first estimation model stored in the storage unit 235. The estimation unit 237 also inputs input data for estimating the logarithmically transformed even components to the second estimation model stored in the storage unit 235. The estimation unit 237 outputs the logarithmically transformed odd components output from the first estimation model and the logarithmically transformed even components output from the second estimation model to the second calculation unit 238. For example, the estimation model (first estimation model/second estimation model) is a multiple regression model constructed using the feature amount selected by the Leave-one-subject-out LASSO method.

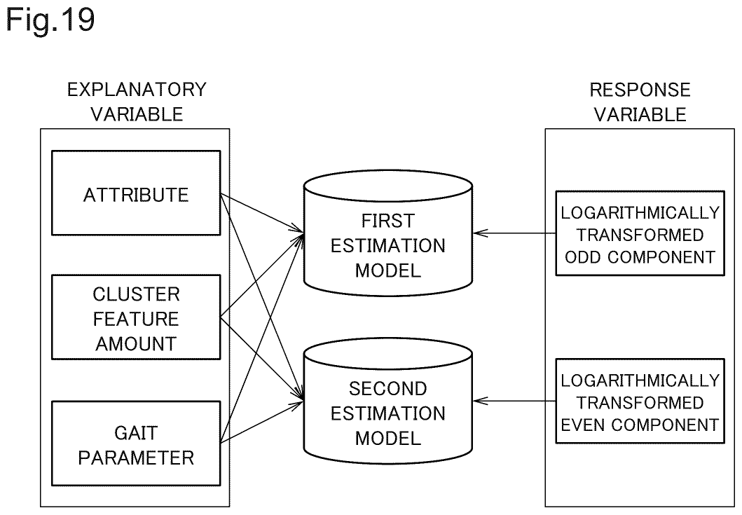

FIG. 19 is a conceptual diagram for explaining an example of learning of an estimation model (first estimation model/second estimation model) used for estimation of frequency components (odd components/even components) used for calculation of a harmonic index. The first estimation model is used for estimation of odd components. The second estimation model is used for estimation of even components. In the example of FIG. 19, an example in which the logarithmically transformed odd components/even components are estimated will be described. The first estimation model and the second estimation model may be configured to estimate odd components/even components that are not logarithmically transformed, rather than logarithmically transformed odd components/even components.

For learning of the estimation model (first estimation model/second estimation model), explanatory variables and response variables related to a plurality of subjects are used. As the explanatory variable, the second feature amounts generated based on the attribute of the subject, the cluster feature amount generated according to the walking of the subject, and the gait parameters are used. As the explanatory variable, the first feature amounts including the cluster feature amounts and the gait parameters generated according to the walking of the subject may be used.

As the response variable, frequency components (odd components/even components) based on the measured value of the spatial acceleration related to the waist are used. For example, the measured value of the spatial acceleration related to the waist is measured by the IMU attached to the waist. In the example of FIG. 19, frequency components (odd components/even components) in each of the vertical direction, the traveling direction, and the left-right direction are used as response variables.

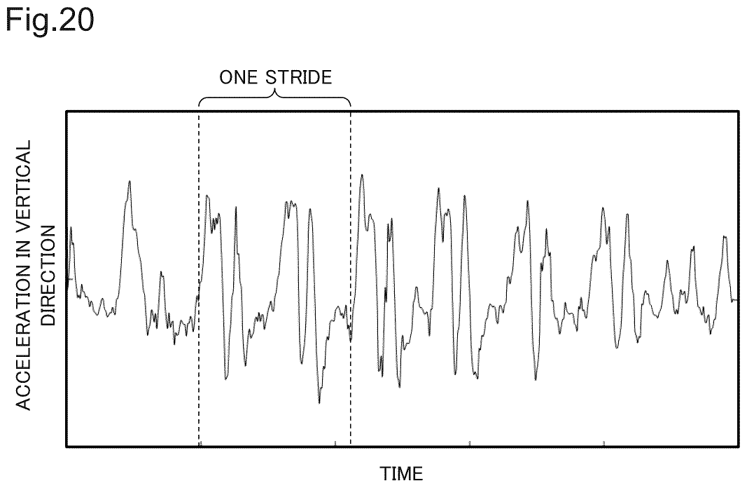

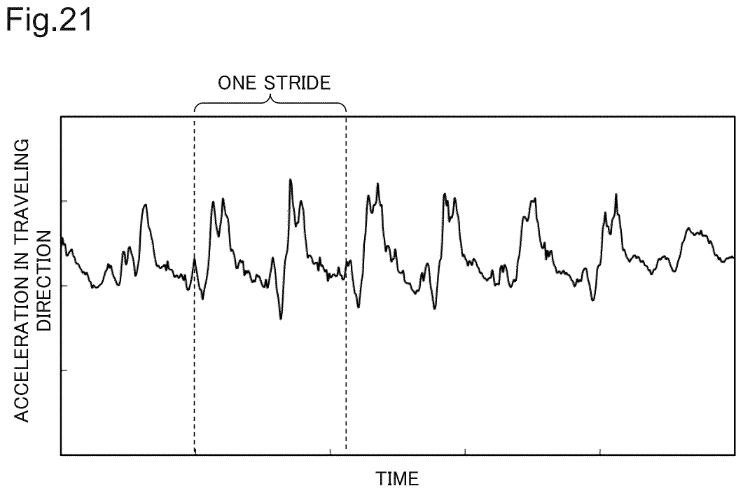

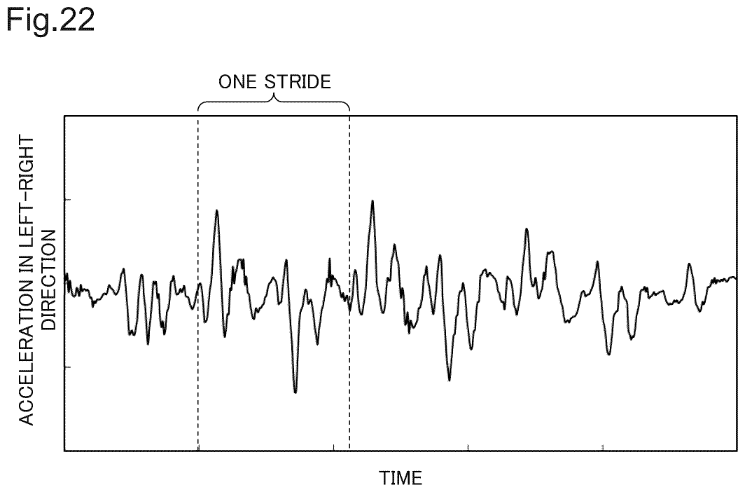

FIGS. 20 to 22 are graphs illustrating an example of time-series data of vertical-direction acceleration, traveling-direction acceleration, and horizontal-direction acceleration of the waist. FIG. 20 is a graph illustrating an example of time-series data of vertical-direction acceleration of the waist. FIG. 21 is a graph illustrating an example of time-series data of the traveling-direction acceleration of the waist. FIG. 22 is a graph illustrating an example of time-series data of the left-right direction acceleration of the waist. Frequency components (odd components/even components) obtained by performing Fourier transform on each of the vertical-direction acceleration, the traveling-direction acceleration, and the horizontal-direction acceleration of the waist for one stride is used as response variables.

Regarding learning of the first estimation model, odd components among the frequency components obtained by performing Fourier transform on time-series data of each of the vertical-direction acceleration, the traveling-direction acceleration, and the horizontal-direction acceleration of the waist are used as response variables. Regarding learning of the second estimation model, even components among the frequency components obtained by performing Fourier transform on time-series data of each of the vertical-direction acceleration, the traveling-direction acceleration, and the horizontal-direction acceleration of the waist are used as response variables. In the example of FIG. 19, the logarithmically transformed frequency components (odd components/even components) are used as the response variables. For example, the learning of the first estimation model and the second estimation model may be performed such that the power sum of the frequency components (odd components/even components) is estimated instead of the frequency components (odd components/even components).

For example, the estimation model (first estimation model/second estimation model) is constructed by learning using a linear regression algorithm. For example, the estimation model (first estimation model/second estimation model) is constructed by learning using a support vector machine (SVM) algorithm. For example, the estimation model (first estimation model/second estimation model) is constructed by learning using a Gaussian process regression (GPR) algorithm. For example, the estimation model (first estimation model/second estimation model) is constructed by learning using a random forest (RF) algorithm. The estimation model (first estimation model/second estimation model) may be constructed by unsupervised learning that classifies subjects who are generation sources of the feature amount data according to the feature amount data. The algorithm used for learning the estimation model (first estimation model/second estimation model) is not particularly limited.

The estimation model (first estimation model/second estimation model) may be constructed by learning using the gait waveform data (sensor data) for one gait cycle as an explanatory variable. For example, the estimation model may be constructed by supervised learning in which the gait waveform data of the acceleration in the three axial directions, the angular velocity about the three axes, and the angles (posture angles) about the three axes is used as explanatory variables and the frequency components (odd components/even components) to be estimated are used as objective variables.

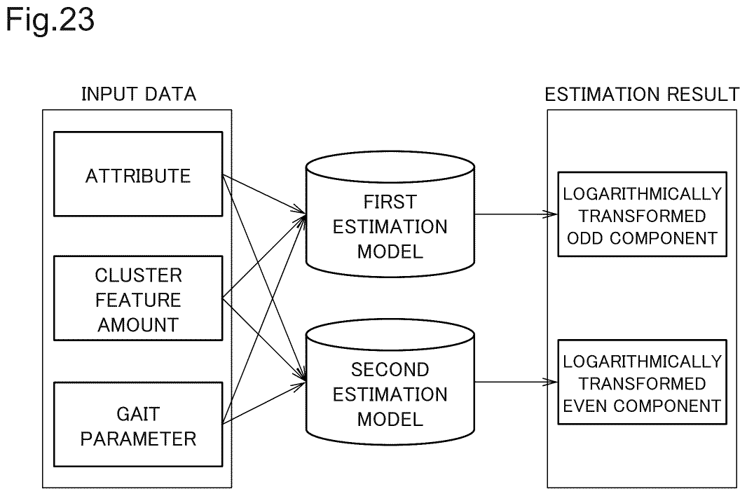

FIG. 23 is a conceptual diagram illustrating an example of estimating a harmonic index of a subject (user) using an estimation model (first estimation model/second estimation model). The estimation model (first estimation model/second estimation model) is a model constructed in advance by learning illustrated in FIG. 19. In the example of FIG. 23, the second feature amount generated based on the attribute of the subject, the cluster feature amount generated according to the walking of the subject, and the gait parameters is used as the input data. In the case of the estimation model generated using the first feature amounts including the cluster feature amounts and the gait parameters, the cluster feature amounts and the gait parameters are used as the input data. The estimation model outputs frequency components (odd components/even components) used for calculation of the harmonic index according to input of input data. In the example of FIG. 23, the logarithmically transformed odd components are output from the first estimation model as an estimation result. In the example of FIG. 23, the logarithmically transformed even components are output from the second estimation model as the estimation result. When one of the odd components and the even components is used, one of the first estimation model and the second estimation model may be used.

The second calculation unit 238 acquires the odd components and the even components estimated by the estimation unit 237. The second calculation unit 238 calculates a power sum for one gait cycle for each of the estimated odd components and even components. The second calculation unit 238 calculates the harmonic index by calculating the ratio between the power sum of the odd components and the power sum of the even components in the vertical direction. The second calculation unit 238 calculates a ratio between the power sum of the odd components and the power sum of the even components in the traveling direction to calculate the harmonic index. The second calculation unit 238 calculates the harmonic index by calculating the ratio of the power sum of the even components and the power sum of the odd components in the left-right direction.

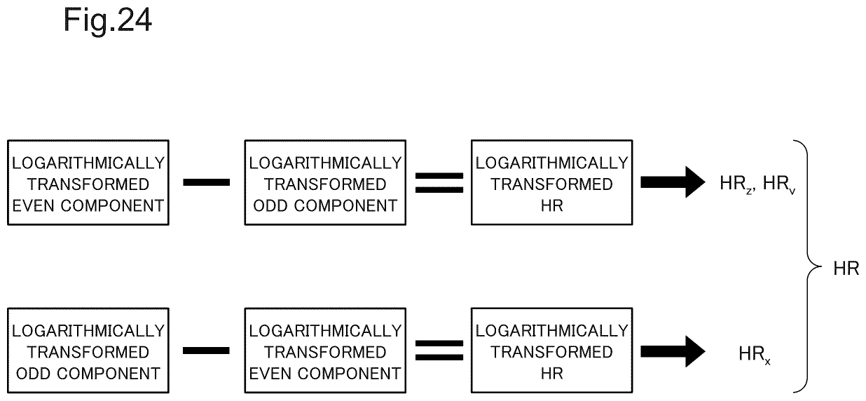

FIG. 24 is a conceptual diagram illustrating an example of calculating the harmonic index HR using logarithmically transformed frequency components (odd components/even components). The second calculation unit 238 subtracts the logarithmically transformed odd components (power sum) from the logarithmically transformed even components (power sum) in the vertical direction and the traveling direction to calculate the logarithmically transformed harmonic index HR. The second calculation unit 238 exponentially converts the logarithmically transformed harmonic index HR in the vertical direction and the traveling direction to calculate the harmonic index (HR_{z}, HR_{y}). The second calculation unit 238 subtracts the logarithmically transformed even components (power sum) from the logarithmically transformed odd components (power sum) in the left-right direction to calculate the logarithmically transformed harmonic index HR. The second calculation unit 238 exponentially converts the logarithmically transformed harmonic index HR in the left-right direction to calculate the harmonic index (HRₓ).

The output unit 239 outputs a calculation result (estimation result) of the harmonic index by the second calculation unit 238. For example, the output unit 239 displays the estimation result of the harmonic index on the screen of the mobile terminal of the subject (user). For example, the output unit 239 outputs the estimation result to an external system or the like that uses the estimation result. The use of the harmonic index output from the harmonic index estimation device 23 is not particularly limited.

For example, the harmonic index estimation device 23 is connected to an external system or the like built in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the harmonic index estimation device 23 is connected to a mobile terminal via a wire such as a cable. For example, the harmonic index estimation device 23 is connected to a mobile terminal via wireless communication. For example, the harmonic index estimation device 23 is connected to a mobile terminal via a wireless communication function (not illustrated) conforming to a standard such as Bluetooth (registered trademark) or WiFi (registered trademark). The communication function of the harmonic index estimation device 23 may conform to a standard other than Bluetooth (registered trademark) or WiFi (registered trademark). The estimation result of the harmonic index may be used by an application installed in the mobile terminal. In that case, the mobile terminal executes processing using the estimation result by application software or the like installed in the mobile terminal.

### Learning Example

Next, a learning example of an estimation model used for estimation of a harmonic index by the harmonic index estimation device 23 will be described with reference to a verification result related to a correlation between frequency components (even components/odd components) used for estimation of the harmonic index and feature amount data. Hereinafter, a verification example performed on forty-five subjects will be described. In the following verification example, the correlation between the measured value and the estimation value of the harmonic index of walking was verified. In the present verification example, a subject wearing a smart apparel and a shoe on which the measurement device 20 is mounted was allowed to walk twice on a straight path of 5 m. An IMU that measures a spatial acceleration and a spatial angular velocity is mounted on the waist of the smart apparel. The measured values were derived using the measured values of the spatial acceleration and the spatial angular velocity of the waist of the subject. The prediction value is an estimation value estimated using sensor data measured by the measurement device 20 mounted on the shoe worn by the subject at the same time as the measurement of the measured value. The correlation between the measured value and the estimation value was evaluated by a value of an intra-class correlation coefficient (ICC). As the intra-class correlation coefficient ICC, an intra-class correlation coefficient ICC(2,k) was used in order to evaluate inter-examiner reliability.

### Vertical Direction

Regarding the estimation of the frequency components (odd components/even components) for estimating the harmonic index in the vertical direction, the average value or difference of the first feature amounts and the gait parameters of both feet is used. Regarding the estimation of the odd components, a difference of the gait parameters of both feet is used. Regarding the estimation of the even components, a difference between the first feature amounts and the gait parameters of both feet is used. Regarding the vertical direction, two methods (first example/second example) will be described.

### FIRST EXAMPLE

A first example is an example in which the average value/difference of the first feature amounts (cluster feature amounts) and the gait parameters are used for estimation. Regarding the traveling direction and the left-right direction, estimation using the first example will also be described.

A plurality of gait parameters is used for estimation of the frequency components (odd components) for estimating the harmonic index in the vertical direction. For example, a difference of both feet with respect to each of the dorsiflexion peak, the pronation/supination angle at the time of touching the ground, DST1, the swinging foot lift height, the load time at the maximum acceleration, and the kicking time is used as the second feature amount.

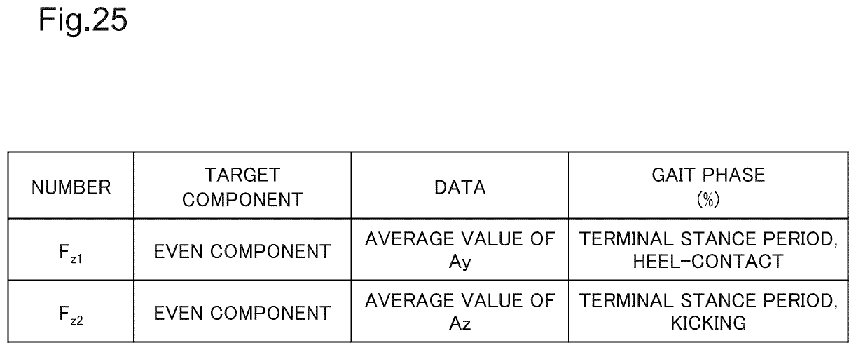

The second feature amount derived from the first feature amount is used for estimation of the frequency components (even components) for estimating the harmonic index in the vertical direction. FIG. 25 is a table summarizing an example of second feature amounts derived from the first feature amounts to be used for estimation of frequency components (even components) for estimating the harmonic index in the vertical direction. Regarding the estimation of the frequency components (even components) in the vertical direction, the average value of both feet of the traveling-direction acceleration A_{y} and the vertical-direction acceleration A_{z} is used as the second feature amount. Regarding the traveling-direction acceleration A_{y}, the second feature amount F_{z1} in the terminal stance period and the heel-contact is used for estimation. Regarding the vertical-direction acceleration A_{z}, the second feature amount F_{z2} in the terminal stance period and the kicking is used for estimation.

A plurality of gait parameters is used for estimation of frequency components (even components) for estimating the harmonic index in the vertical direction. For example, an average value of both feet of each of the dorsiflexion peak and DST1 is used as the second feature amount.

The estimation value of the harmonic index in the vertical direction is calculated by calculating the ratio of the power sum of the even components and the power sum of the odd components. In this verification, regarding the estimation of the harmonic index in the vertical direction, the intra-class correlation coefficient ICC(2,k) between the measured value and the estimation value was 0.7804.

### SECOND EXAMPLE

The second example is an example in which the average value/difference of the gait parameters is used for estimation. In the second example, the first feature amount (cluster feature amount) is not used for estimation. The second example is an example in which frequency components (odd components/even components) based on the spatial acceleration of the waist are estimated using frequency components obtained by performing Fourier transform on time-series data of the spatial acceleration of the foot portion. Frequency components (also referred to as frequency feature amounts) based on the spatial acceleration of the foot portion are selected according to the number of vibrations per gait phase. Frequency components in which the number of vibrations per gait phase is n are expressed as frequency components of n vibrations (n is a natural number). In the following example, a combination of frequency components having the maximum correlation is selected by verifying the combinations of the frequency components of 1 to 20 vibrations in a round-robin manner.

In the estimation of the frequency components (odd components), frequency components of 1, 3, and 5 vibrations among frequency components based on the vertical-direction acceleration of the foot are used for the estimation. In the estimation of the frequency components (odd components), a plurality of gait parameters is used for estimation. For example, a difference of both feet with respect to each of the dorsiflexion peak, the pronation/supination angle at the time of touching the ground, DST1, the swinging foot lift height, the load time at the maximum acceleration, and the kicking time is used for estimation.

In the estimation of the frequency components (even components), frequency components of 1, 3, 4, 5, and 6 vibrations among frequency components based on the vertical-direction acceleration of the foot are used for the estimation. In the estimation of the frequency components (odd components), a plurality of gait parameters is used for estimation. For example, the average value of both feet for each of the dorsiflexion peak and DST1 is used for estimation.

In the second example, the intra-class correlation coefficient ICC(2,k) between the measured value and the estimation value was 0.7756. In the second example, the correlation between the measured value and the estimation value was equivalent to that in the first example even when the second feature amount derived from the cluster feature amount was not used. According to the second example, the harmonic index can be estimated with a smaller number of pieces of data than the first example. The method of the second example can also be applied to a traveling direction and a left-right direction described later.

### Traveling Direction

Regarding the estimation of the frequency components (odd components/even components) for estimating the harmonic index in the traveling direction, the average value or difference of the first feature amounts and the gait parameters of both feet is used. Regarding the estimation of the odd components, a difference of the gait parameters of both feet is used. Regarding the estimation of the even components, a difference between the first feature amounts and the gait parameters of both feet is used.

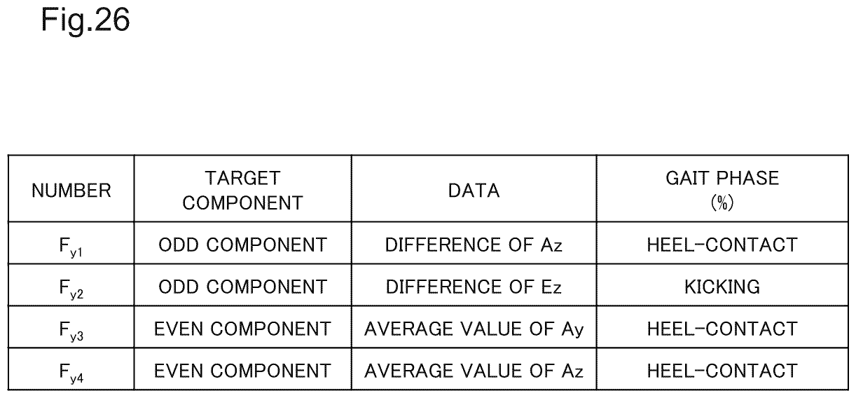

The second feature amount derived from the first feature amount is used for estimation of the frequency components (odd components/even components) for estimating the harmonic index in the traveling direction. FIG. 26 is a table summarizing an example of the second feature amount derived from the first feature amount to be used for the estimation of the frequency components (odd components/even components) for estimating the harmonic index in the traveling direction.

Regarding the estimation of the frequency components (odd components) in the traveling direction, the average value of both feet of the vertical-direction acceleration A_{z} and the angle E_{z} about the vertical axis is used as the second feature amount. Regarding the vertical-direction acceleration A_{z}, the second feature amount F_{y1} at the heel-contact is used for estimation. Regarding the angle E_{z} about the vertical axis, the second feature amount F_{y2} at the kicking is used for estimation.

A plurality of gait parameters is used for estimation of frequency components (odd components) in the traveling direction. For example, a difference of both feet with respect to each of the plantarflexion angle, the pronation/supination angle at the time of touching the ground, DST2, the maximum dorsiflexion/plantarflexion angular velocity in the swing phase, and the kicking time is used as the second feature amount.

Regarding the estimation of the frequency components (even components) in the traveling direction, the average value of both feet of the traveling-direction acceleration A_{y} and the vertical-direction acceleration A_{z} is used as the second feature amount. Regarding the traveling-direction acceleration A_{y}, the second feature amount F_{y3} at the heel-contact is used for estimation. Regarding the vertical-direction acceleration A_{z}, the second feature amount F_{y4} at the heel-contact is used for estimation.

A plurality of gait parameters is used for estimation of frequency components (even components) in the traveling direction. For example, an average value of both feet of each of the stride length, the dorsiflexion peak, the pronation/supination angle at the time of touching the ground, DST1, and the maximum speed in the swing phase is used as the second feature amount.

The estimation value of the harmonic index in the traveling direction is calculated by calculating the ratio of the power sum of the even components and the power sum of the odd components. In this verification, regarding the estimation of the harmonic index in the traveling direction, the intra-class correlation coefficient ICC(2,k) between the measured value and the estimation value was 0.6671.

### Left-Right Direction

Regarding the estimation of the frequency components (odd components/even components) for estimating the harmonic index in the left-right direction, the average value or difference of the first feature amounts and the gait parameters of both feet is used. Regarding the estimation of the odd components, the average value of the first feature amounts and the gait parameters of both feet is used. Regarding the estimation of the even components, a difference in the gait parameters of both feet is used.

A plurality of gait parameters is used for estimation of the frequency components (odd components) for estimating the harmonic index in the left-right direction. For example, a difference of both feet of each of the plantarflexion peak, the foot angle, the DST1, the maximum speed during the swing phase, the heel-contact time, and the kicking time is used as the second feature amount.

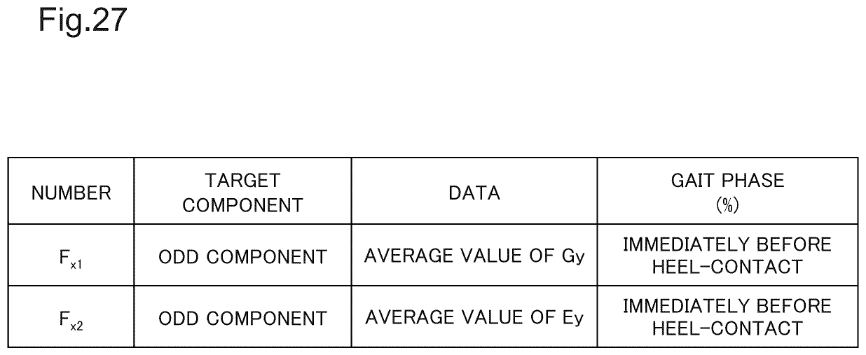

The second feature amount derived from the first feature amount is used for estimation of the frequency components (odd components) for estimating the harmonic index in the left-right direction. FIG. 27 is a table summarizing an example of the second feature amount derived from the first feature amount to be used for the estimation of the frequency components (odd components) for estimating the harmonic index in the left-right direction. Regarding the estimation of the frequency components (odd components) in the left-right direction, the average value of both feet of the angular velocity G_{y} about the axis of travel and the angle E_{y} about the axis of travel is used as the second feature amount. Regarding the angular velocity G_{y} about the axis of travel, the second feature amount Fₓ₁ immediately before the heel-contact is used for estimation. Regarding the angle E_{y} about the axis of travel, the second feature amount Fₓ₂ immediately before the heel-contact is used for estimation.

A plurality of gait parameters is used for estimation of frequency components (even components) for estimating the harmonic index in the left-right direction. For example, the average value of the plantarflexion peak, the foot angle, the foot swing length, the maximum speed in the swing phase, the heel-contact time, and the kicking time is used as the second feature amount.

The estimation value of the harmonic index in the left-right direction is calculated by calculating the ratio of the power sum of the odd components and the power sum of the even components. In this verification, regarding the estimation of the harmonic index in the left-right direction, the intra-class correlation coefficient ICC(2,k) between the measured value and the estimation value was 0.7488.

### Operation

Next, an operation of the estimation system 2 will be described with reference to the drawings. Here, an operation of the harmonic index estimation device 23 included in the estimation system 2 will be described. Since the measurement device 20 operates similarly to the measurement device 10 of the first example embodiment, the description thereof is omitted.

### Harmonic Index Estimation Device

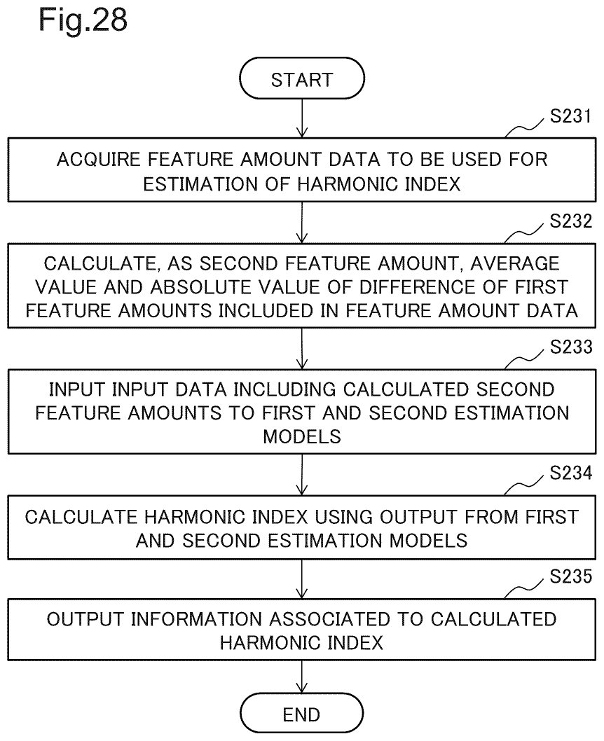

FIG. 28 is a flowchart for explaining an operation of the harmonic index estimation device 23. In the description according to the flowchart of FIG. 28, the harmonic index estimation device 23 will be described as the subject of an operation. The flowchart of FIG. 28 relates to a case (first example) exemplified in the estimation of the frequency components (odd components) for estimating the harmonic index. In FIG. 28, first, the harmonic index estimation device 23 acquires the feature amount data to be used for the estimation of the harmonic index from the measurement device 20 (step S231).

Next, the harmonic index estimation device 23 calculates, as the second feature amount, the average value and the absolute value of the difference of the first feature amounts included in the acquired feature amount data (step S232).

Next, the harmonic index estimation device 23 inputs input data including the calculated second feature amount to the first estimation model/second estimation model that estimates the harmonic index (step S233). The harmonic index estimation device 23 inputs input data to be used for estimation of odd components to the first estimation model. The harmonic index estimation device 23 inputs input data to be used for estimation of even components to the second estimation model.

Next, the harmonic index estimation device 23 calculates a harmonic index using the output (estimation value) from the first estimation model/the second estimation model (step S234). The first estimation model outputs an estimation result related to odd components. The second estimation model outputs the estimation result related to even components. The harmonic index estimation device 23 calculates the harmonic index using the odd components and the even components in each of the vertical direction, the traveling direction, and the left-right direction.

Next, the harmonic index estimation device 23 outputs information corresponding to the calculated harmonic index (step S235). For example, the harmonic index is output to a terminal device (not illustrated) carried by the user. For example, information corresponding to the harmonic index is output to a system that executes processing using the information.

### SECOND EXAMPLE

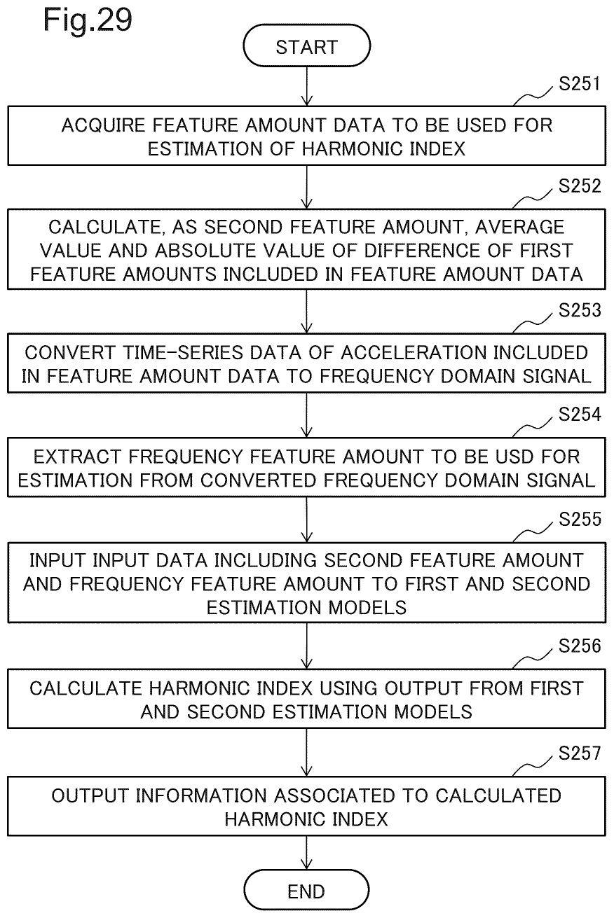

Next, a second example exemplified in the estimation of the frequency components (odd components) for estimating the harmonic index in the vertical direction will be described with reference to a flowchart. FIG. 29 is a flowchart for explaining the second example. In the description according to the flowchart of FIG. 29, the harmonic index estimation device 23 will be described as the subject of an operation.

In FIG. 29, first, the harmonic index estimation device 23 acquires feature amount data to be used for estimation of the harmonic index from the measurement device 20 (step S251).

Next, the harmonic index estimation device 23 calculates, as the second feature amount, the average value and the absolute value of the difference of the first feature amounts included in the acquired feature amount data (step S252).

Next, the harmonic index estimation device 23 converts the time-series data of the acceleration included in the feature amount data into a frequency domain signal by performing Fourier transform (step S253).

Next, the harmonic index estimation device 23 extracts a frequency feature amount to be used for estimation from the converted frequency domain signal (step S254). For example, the harmonic index estimation device 23 extracts a frequency feature amount to be used for estimation based on the number of vibrations per gait phase.

Next, the harmonic index estimation device 23 inputs input data including the second feature amount and the frequency feature amount to the first estimation model/second estimation model (step S255). The harmonic index estimation device 23 inputs input data to be used for estimation of odd components to the first estimation model. The harmonic index estimation device 23 inputs input data to be used for estimation of even components to the second estimation model.

Next, the harmonic index estimation device 23 calculates a harmonic index using the output (estimation value) from the first estimation model/the second estimation model (step S256). The first estimation model outputs an estimation result related to odd components. The second estimation model outputs the estimation result related to even components. The harmonic index estimation device 23 calculates the harmonic index using the odd components and the even components in each of the vertical direction, the traveling direction, and the left-right direction.

Next, the harmonic index estimation device 23 outputs information corresponding to the calculated harmonic index (step S257). For example, the harmonic index is output to a terminal device (not illustrated) carried by the user. For example, information corresponding to the harmonic index is output to a system that executes processing using the information.

As described above, the estimation system of the present example embodiment includes the measurement device and the harmonic index estimation device. The measurement device is installed on the footwear of the subject who is an estimation target of the harmonic index that is the index related to the movement of the waist. The measurement device includes a sensor and a feature amount data generation unit. The sensor measures a spatial acceleration and a spatial angular velocity. The sensor generates sensor data related to the movement of the foot using the measured spatial acceleration and spatial angular velocity. The sensor outputs the generated sensor data. The feature amount data generation unit acquires time-series data of sensor data including features of a gait. The feature amount data generation unit extracts gait waveform data for one gait cycle from the time-series data of the sensor data. The feature amount data generation unit normalizes the extracted gait waveform data. The feature amount data generation unit extracts, from the normalized gait waveform data, a feature amount to be used for estimation of the harmonic index from a gait phase cluster including at least one temporally consecutive gait phase. The feature amount data generation unit generates feature amount data including the extracted feature amount. The feature amount data generation unit outputs the generated feature amount data to the harmonic index estimation device.

The harmonic index estimation device includes a communication unit, a first calculation unit, a storage unit, an estimation unit, a second calculation unit, and an output unit. The communication unit acquires feature amount data including a feature amount extracted from the gait waveforms of the spatial acceleration and the spatial angular velocity included in the sensor data related to the movement of the foot of the subject and used for estimation of the harmonic index related to smoothness of the movement of the waist. The first calculation unit calculates, as the second feature amount, an average value and a difference of the first feature amounts to be used for estimation of the harmonic index among the first feature amounts for both feet of the subject. The storage unit stores an estimation model including a first estimation model that outputs odd components according to an input of a second feature amount and a second estimation model that outputs even components according to an input of a second feature amount. The even components are frequency components corresponding to an element during the gait cycle among frequency components obtained by performing Fourier transform on the time-series data of the spatial acceleration of the waist. The odd components are frequency components that do not correspond to an element during the gait cycle among frequency components obtained by performing Fourier transform on the time-series data of the spatial acceleration of the waist. The estimation unit inputs the second feature amount to the first estimation model to estimate odd components. The estimation unit inputs the second feature amount to the second estimation model to estimate even components. The second calculation unit calculates the harmonic index of the subject using the estimated power sum of the odd components and the even components for one gait cycle. The output unit outputs information corresponding to the harmonic index of the subject.

In the present example embodiment, frequency components (odd components/even components) obtained by performing Fourier transform on the time-series data of the spatial acceleration of the waist is estimated using the feature amount extracted from the sensor data related to the movement of the foot of the subject. In the present example embodiment, the harmonic index related to the smoothness of the movement of the waist of the subject is calculated using the estimated frequency components (odd components/even components). Therefore, according to the present example embodiment, it is possible to easily estimate the harmonic index related to the smoothness of the movement of the waist with high accuracy in daily life.

### Third Example Embodiment

Next, a harmonic index estimation device according to a third example embodiment will be described with reference to the drawings. The harmonic index estimation device of the present example embodiment has a simplified configuration of the harmonic index estimation devices of the first and second example embodiments.

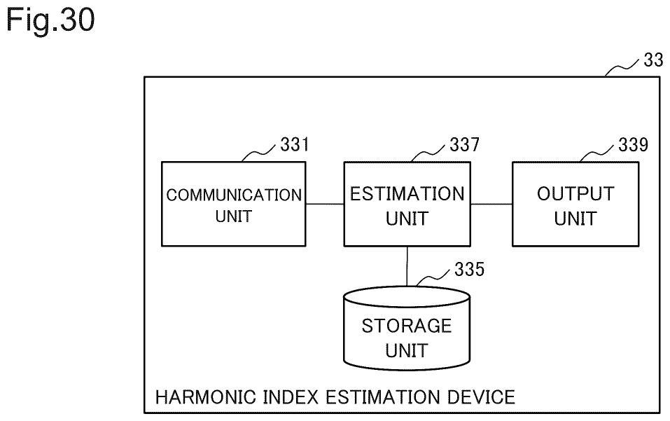

FIG. 30 is a block diagram illustrating an example of a configuration of a harmonic index estimation device 33 according to the present example embodiment. The harmonic index estimation device 33 includes a communication unit 331, a storage unit 335, an estimation unit 337, and an output unit 339.

The communication unit 331 acquires feature amount data including a feature amount extracted from the gait waveforms of the spatial acceleration and the spatial angular velocity included in the sensor data related to the movement of the foot of the subject and used for estimation of the harmonic index related to smoothness of the movement of the waist. The storage unit 335 stores an estimation model that outputs an estimation value related to the harmonic index, according to an input of a feature amount included in the feature amount data. The estimation unit 337 inputs the feature amount included in the acquired feature amount data to the estimation model, and estimates the harmonic index of the subject according to the estimation value related to the harmonic index output from the estimation model. The output unit 339 outputs information corresponding to the harmonic index of the subject.

In the present example embodiment, the harmonic index related to smoothness of the movement of the waist of the subject is estimated using the feature amount extracted from the sensor data related to the movement of the foot of the subject. Therefore, according to the present example embodiment, it is possible to easily estimate the harmonic index related to the smoothness of the movement of the waist with high accuracy in daily life.

### Hardware

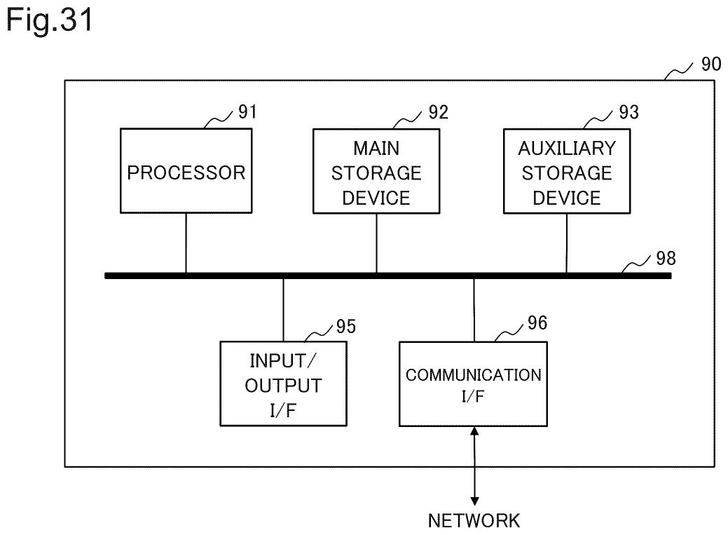

Here, a hardware configuration for executing the processing according to each example embodiment of the present disclosure will be described using an information processing device (computer) 90 of FIG. 31 as an example. The information processing device 90 in FIG. 31 is a configuration example for executing the processing of each example embodiment, and does not limit the scope of the present disclosure.

As illustrated in FIG. 31, the information processing device 90 includes a processor 91, a main storage device 92, an auxiliary storage device 93, an input/output interface 95, and a communication interface 96. In FIG. 31, the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the processing of each example embodiment. The processor 91 executes the program developed in the main storage device 92. The processor 91 executes the processing according to each example embodiment by executing the program.

The main storage device 92 has an area in which a program is developed. A program stored in the auxiliary storage device 93 or the like is developed in the main storage device 92 by the processor 91. The main storage device 92 is, for example, a volatile memory such as a dynamic random access memory (DRAM). A nonvolatile memory such as a magnetoresistive random access memory (MRAM) may be configured and added as the main storage device 92.

The auxiliary storage device 93 stores various types of data such as programs. The auxiliary storage device 93 is a local disk such as a hard disk or a flash memory. Various types of data may be stored in the main storage device 92, and the auxiliary storage device 93 may be omitted.

The input/output interface 95 is an interface for connecting the information processing device 90 and a peripheral device based on a standard or a specification. The communication interface 96 is an interface for connecting to an external system or device through a network such as the Internet or an intranet based on a standard or a specification. The input/output interface 95 and the communication interface 96 may be shared as an interface connected to an external device.

Input devices such as a keyboard, a mouse, and a touch panel may be connected to the information processing device 90 as necessary. These input devices are used for inputting information and settings. When a touch panel is used as the input device, a screen having a touch panel function serves as an interface. The processor 91 and the input device are connected via the input/output interface 95.

The information processing device 90 may be provided with a display device for displaying information. In a case where a display device is provided, the information processing device 90 includes a display control device (not illustrated) for controlling display of the display device. The information processing device 90 and the display device are connected via the input/output interface 95.

The information processing device 90 may be provided with a drive device. The drive device mediates reading of data and a program stored in a recording medium and writing of a processing result from the information processing device 90 to the recording medium between the processor 91 and the recording medium (program recording medium). The information processing device 90 and the drive device are connected via the input/output interface 95.

The above is an example of a hardware configuration for enabling the processing according to each example embodiment of the present invention. The hardware configuration of FIG. 31 is an example of a hardware configuration for executing the processing according to each example embodiment, and does not limit the scope of the present invention. A program for causing a computer to execute processing according to each example embodiment is also included in the scope of the present invention.

A program recording medium in which the program according to each example embodiment is recorded is also included in the scope of the present invention. The recording medium can be achieved by, for example, an optical recording medium such as a compact disc (CD) or a digital versatile disc (DVD). The recording medium may be a semiconductor recording medium such as a universal serial bus (USB) memory or a secure digital (SD) card. The recording medium may be a magnetic recording medium such as a flexible disk, or another recording medium. In a case where the program executed by the processor is recorded in the recording medium, the recording medium corresponds to a program recording medium.

The components of the example embodiments may be arbitrarily combined. The components of the example embodiments may be implemented by software. The components of each example embodiment may be implemented by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A harmonic index estimation device comprising:
a memory storing instructions, and
a processor connected to the memory and configured to execute the instructions to:
acquire feature amount data including a feature amount to be used for estimation of a harmonic index related to smoothness of movement of a waist, the feature amount being extracted from a gait waveform of a spatial acceleration and a spatial angular velocity included in sensor data related to movement of a foot of a user;
input the feature amount included in the acquired feature amount data to a machine learning model that outputs an estimation value related to the harmonic index according to an input of the feature amount included in the feature amount data;
estimate a harmonic index of the user according to the estimation value related to the harmonic index output from the machine learning model; and
display information regarding a hospital at which the user can seek medical advice according to the estimation result of the harmonic index of the user on a screen of a mobile terminal used by the user.

2. The harmonic index estimation device according to claim 1, wherein
the machine learning model is trained to output an estimation value related to the harmonic index, according to an input of the first feature amount included in the feature amount data,
the processor is configured to execute the instructions to
acquire the feature amount data including a first feature amount including at least one of gait parameters extracted from a gait waveform of the spatial acceleration and the spatial angular velocity included in the sensor data and a cluster feature amount for each gait phase cluster,
input the first feature amount included in the acquired feature amount data to the machine learning model,
estimate the harmonic index of the user according to the estimation value related to the harmonic index output from the machine learning model.

3. The harmonic index estimation device according to claim 2, wherein
the machine learning model is trained to output an estimation value related to the harmonic index, according to an input of the second feature amount,
the processor is configured to execute the instructions to
calculate, as a second feature amount, an average value and a difference of the first feature amounts to be used for estimation of the harmonic index among the first feature amounts for both feet of the user,
input the calculated second feature amount to the machine learning model, and
estimate the harmonic index of the user according to the estimation value related to the harmonic index output from the machine learning model.

4. The harmonic index estimation device according to claim 3, wherein
the machine learning model is trained to output output an estimation value related to the harmonic index, according to an input of an attribute of the user and the second feature amount,
the processor is configured to execute the instructions to
input the attribute of the user and the second feature amount to the machine learning model, and
estimate the harmonic index of the user according to the estimation value related to the harmonic index output from the machine learning model.

5. The harmonic index estimation device according to claim 3, wherein
the machine learning model includes a first machine learning model trained to output the odd components according to an input of the second feature amount and a second machine learning model trained to output the even components according to an input of the second feature amount,
the processor is configured to execute the instructions to
input the second feature amount to the first machine learning model to estimate the odd components; and
input the second feature amount to the second machine learning model to estimate the even components, and
estimate the harmonic index of the user using a power sum of the estimated odd components and even components for one gait cycle.

6. The harmonic index estimation device according to claim 2, wherein
the machine learning model is trained to output at least one of the harmonic indices in three directions of a traveling direction, a left-right direction, and a vertical direction in one gait cycle as the estimation value related to the harmonic index, according to the input of the first feature amount included in the feature amount data,
the processor is configured to execute the instructions to
input the first feature amount included in the acquired feature amount data to the machine learning model, and
estimate the harmonic index of the user according to at least one of the harmonic indices in the three directions of the traveling direction, the left-right direction, and the vertical direction output from the machine learning model.

7. The harmonic index estimation device according to claim 1, wherein
the processor is configured to execute the instructions to
display recommendation information according to the estimation result of the harmonic index of the user on the screen of the mobile terminal used by the user with content optimized for healthcare application.

8. An estimation system comprising:
the harmonic index estimation device according to claim 1; and
a measurement device including a sensor that measures a spatial acceleration and a spatial angular velocity, and generates the sensor data based on the spatial acceleration and the spatial angular velocity, and configured to generate feature amount data including a feature amount used for estimating a harmonic index using the sensor data.

9. An estimation method executed by a computer, the method comprising:
acquiring feature amount data including a feature amount to be used for estimation of a harmonic index related to smoothness of movement of a waist, the feature amount being extracted from a gait waveform of a spatial acceleration and a spatial angular velocity included in sensor data related to movement of a foot of a user;
inputting the feature amount included in the acquired feature amount data to a machine learning model that outputs an estimation value related to the harmonic index according to an input of the feature amount included in the feature amount data;
estimating a harmonic index of the user according to the estimation value related to the harmonic index output from the machine learning model; and
displaying information regarding a hospital at which the user can seek medical advice according to the estimation result of the harmonic index of the user on a screen of a mobile terminal used by the user.

10. A non-transitory program recording medium recorded with a program causing a computer to perform the following processes:
acquiring feature amount data including a feature amount to be used for estimation of a harmonic index related to smoothness of movement of a waist, the feature amount being extracted from a gait waveform of a spatial acceleration and a spatial angular velocity included in sensor data related to movement of a foot of a user;
inputting the feature amount included in the acquired feature amount data to a machine learning model that outputs an estimation value related to the harmonic index according to an input of the feature amount included in the feature amount data;
estimating a harmonic index of the user according to the estimation value related to the harmonic index output from the machine learning model; and
displaying information regarding a hospital at which the user can seek medical advice according to the estimation result of the harmonic index of the user on a screen of a mobile terminal used by the user.
